SEQUENCE LISTING (55 sequences)

SEQ ID NO: 1 - protein, 214 aa, Artificial Sequence
  Light chain sequence
DIQMTQSPSSLSASVGDRVTITCKASRNIERNLAWYQQKPGQAPELLIYQASRKESGVPDRFSGSRYGTDFTLTISSLQPEDIATYYCQQYSSPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 446 aa, Artificial Sequence
  Factor X heavy chain
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 3 - protein, 450 aa, Artificial Sequence
  Factor IX heavy chain
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 446 aa, Artificial Sequence
  Factor X heavy chain
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 450 aa, Artificial Sequence
  Factor IX heavy chain
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 450 aa, Artificial Sequence
  Factor IX heavy chain
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGK

SEQ ID NO: 7 - protein, 446 aa, Artificial Sequence
  Factor X heavy chain
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVYPPSQEEMTKNQVSLTCLVKGFYPSDIAVEWESNGQPENNYKTTPPVLDSDGSFALVSRLTVDKSRWQEGNVFSCSVMHEALHNHYTQKSLSLSLGK

SEQ ID NO: 8 - protein, 785 aa, Artificial Sequence
  Factor IX heavy chain conjugated to A5
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSGGGGSGGGGSAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 9 - protein, 785 aa, Artificial Sequence
  Factor IX heavy chain conjugated to A5
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGKSGGGGSGGGGSGGGGSAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 10 - protein, 781 aa, Artificial Sequence
  Factor X heavy chain conjugated to A5
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVYPPSQEEMTKNQVSLTCLVKGFYPSDIAVEWESNGQPENNYKTTPPVLDSDGSFALVSRLTVDKSRWQEGNVFSCSVMHEALHNHYTQKSLSLSLGKSGGGGSGGGGSGGGGSAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 11 - protein, 107 aa, Artificial Sequence
  Light chain variable sequence
DIQMTQSPSSLSASVGDRVTITCKASRNIERNLAWYQQKPGQAPELLIYQASRKESGVPDRFSGSRYGTDFTLTISSLQPEDIATYYCQQYSSPPLTFGGGTKVEIK

SEQ ID NO: 12 - protein, 119 aa, Artificial Sequence
  Factor X heavy chain variable region
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSS

SEQ ID NO: 13 - protein, 123 aa, Artificial Sequence
  Factor IX heavy chain variable region
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSS

SEQ ID NO: 14 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 15 - protein, 327 aa, Artificial Sequence
  Stabilised IgG4 heavy chain constant
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 327 aa, Artificial Sequence
  human IgG4 heavy chain constant region with S228P, T366W mutations
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 327 aa, Artificial Sequence
  human IgG4 heavy chain constant region with S228P, T366S, L368A, Y407V mutations
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 18 - protein, 327 aa, Artificial Sequence
  human IgG4 heavy chain constant region with T350V, T366L, K392L, T394W mutations
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVLPPSQEEMTKNQVSLLCLVKGFYPSDIAVEWESNGQPENNYLTWPPVLDSDGSFFLYSRLTVDKSRWQEGNVFSCSVMHEALHNHYTQKSLSLSLGK

SEQ ID NO: 19 - protein, 327 aa, Artificial Sequence
  human IgG4 heavy chain constant region with T350V, L351Y, F405A, Y407V mutations
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVYPPSQEEMTKNQVSLTCLVKGFYPSDIAVEWESNGQPENNYKTTPPVLDSDGSFALVSRLTVDKSRWQEGNVFSCSVMHEALHNHYTQKSLSLSLGK

SEQ ID NO: 20 - protein, 16 aa, Artificial Sequence
  Linker
SGGGGSGGGGSGGGGS

SEQ ID NO: 21 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 22 - protein, 462 aa, Homo sapiens [sequence omitted]

SEQ ID NO: 23 - protein, 488 aa, Homo sapiens
MGRPLHLVLLSASLAGLLLLGESLFIRREQANNILARVTRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 24 - protein, 6 aa, Artificial Sequence
  GS6 Linker
SGGGGS

SEQ ID NO: 25 - protein, 31 aa, Artificial Sequence
  GS31 Linker
SGGGGSGGGGSGGGGSGGGGSGGGGSGGGGS

SEQ ID NO: 26 - protein, 319 aa, Artificial Sequence
  E5-mutant of annexin A5
AQVLRGTVTDFPGFDEEADAETLEKAMEGLGTDEESILTLLTSRSNAQRQEISAAFETLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEEFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 27 - protein, 318 aa, artificial
  Lactadherin C1C2 sequence (aka MFG-E8)
CVEPLGMENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 28 - protein, 9 aa, artificial
  PSP1 peptide
CLSYYPSYC

SEQ ID NO: 29 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 30 - protein, 305 aa, Artificial Sequence
  Truncated annexin A1
FNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 31 - protein, 785 aa, Artificial Sequence
  Factor IX heavy chain conjugated to E5 mutant of annexin A5
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSGGGGSGGGGSAQVLRGTVTDFPGFDEEADAETLEKAMEGLGTDEESILTLLTSRSNAQRQEISAAFETLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEEFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 32 - protein, 771 aa, Artificial Sequence
  Factor IX heavy chain conjugated to truncated annexin A1
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSGGGGSGGGGSFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 33 - protein, 786 aa, Artificial Sequence
  Factor IX heavy chain conjugated to truncated annexin A1
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSGGGGSGGGGSGGGGSGGGGSGGGGSFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 34 - protein, 450 aa, Artificial Sequence
  amino acid sequence of Factor IX heavy chain A10
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGRTKPWGYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 123 aa, Artificial Sequence
  heavy chain VH amino acid sequence of Factor IX A10
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGRTKPWGYYFDYWGQGTLVTVSS

SEQ ID NO: 36 - protein, 785 aa, Artificial Sequence
  amino acid sequence of Factor IX heavy chain A10 fused to annexin A5
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGRTKPWGYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSGGGGSGGGGSAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 37 - protein, 450 aa, Artificial Sequence
  amino acid sequence of Factor IX heavy chain B2
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGRIKTWGYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 123 aa, Artificial Sequence
  heavy chain VH amino acid sequence of Factor IX B2
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGRIKTWGYYFDYWGQGTLVTVSS

SEQ ID NO: 39 - protein, 785 aa, Artificial Sequence
  amino acid sequence of Factor IX heavy chain B2 fused to annexin A5
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGRIKTWGYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSGGGGSGGGGSAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 40 - protein, 450 aa, Artificial Sequence
  amino acid sequence of Factor IX heavy chain C12
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGRKKGWHFYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 123 aa, Artificial Sequence
  heavy chain VH amino acid sequence of Factor IX C12
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGRKKGWHFYFDYWGQGTLVTVSS

SEQ ID NO: 42 - protein, 785 aa, Artificial Sequence
  amino acid sequence of Factor IX heavy chain C12 fused to annexin A5
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGRKKGWHFYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSGGGGSGGGGSGGGGSAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 43 - protein, 5 aa, Artificial Sequence
  heavy chain VH CDR1 amino acid sequence of Factor IX
YYDIQ

SEQ ID NO: 44 - protein, 17 aa, Artificial Sequence
  heavy chain VH CDR2 amino acid sequence of Factor IX
SISPSGQSTYYRREVKG

SEQ ID NO: 45 - protein, 14 aa, Artificial Sequence
  heavy chain VH CDR3 amino acid sequence of Factor IX
RTGREYGGGWYFDY

SEQ ID NO: 46 - protein, 14 aa, Artificial Sequence
  heavy chain VH CDR3 amino acid consensus sequence of Factor IX
  X (5)..(5): T or I or K or E
  X (6)..(6): K or Y
  X (7)..(7): P or T or G
  X (8)..(8): W or G
  X (9)..(9): G or H
  X (10)..(10): Y or F or W
RTGRXXXXXXYFDY

SEQ ID NO: 47 - protein, 11 aa, Artificial Sequence
  light chain VL CDR1 amino acid sequence of Factor IX
KASRNIERNLA

SEQ ID NO: 48 - protein, 7 aa, Artificial Sequence
  light chain VL CDR2 amino acid sequence of Factor IX
QASRKES

SEQ ID NO: 49 - protein, 9 aa, Artificial Sequence
  light chain VL CDR3 amino acid sequence of Factor IX
QQYSSPPLT

SEQ ID NO: 50 - protein, 123 aa, Artificial Sequence
  heavy chain VH amino acid consensus sequence of Factor IX
  X (103)..(103): T or I or K or E
  X (104)..(104): K or Y
  X (105)..(105): P or T or G
  X (106)..(106): W or G
  X (107)..(107): G or H
  X (108)..(108): Y or F or W
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGRXXXXXXYFDYWGQGTLVTVSS

SEQ ID NO: 51 - protein, 14 aa, Artificial Sequence
  heavy chain VL CDR3 amino acid sequence of Factor IX A10
RTGRTKPWGYYFDY

SEQ ID NO: 52 - protein, 14 aa, Artificial Sequence
  heavy chain VL CDR3 amino acid sequence of Factor IX B2
RTGRIKTWGYYFDY

SEQ ID NO: 53 - protein, 14 aa, Artificial Sequence
  heavy chain VH CDR3 amino acid sequence of Factor IX C12
RTGRKKGWHFYFDY

SEQ ID NO: 54 - protein, 453 aa, Artificial Sequence
  amino acid sequence of anti-Factor IX antibody heavy chain ATG16028
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPRCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGK

SEQ ID NO: 55 - protein, 788 aa, Artificial Sequence
  amino acid sequence of anti-Factor IX antibody heavy chain ATG16028 fused to Annexin A5
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCRKTHTCPRCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSRDELTKNQVSLSCLVKGFYPSDIAVEWESNGQPENNYKTTVPVLDSDGSFRLASYLTVDKSRWQQGNVFSCSVMHEALHNHYTQKSLSLSPGKSGGGGSGGGGSGGGGSAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

(19) United States
(12) Patent Application Publication
(10) Pub. No.: US 2019/0248920 A1
(43) Pub. Date: Aug. 15, 2019

(54) COAGULATION FACTOR BINDING PROTEINS AND USES THEREOF

(71) Applicant: CSL LIMITED, Parkville, Victoria (AU)

(72) Inventors: Peter SCHMIDT, Parkville, Victoria (AU); Chao-Guang CHEN, Parkville, Victoria (AU); Con PANOUSIS, Parkville, Victoria (AU); Louis FABRI, Parkville, Victoria (AU)

(21) Appl. No.: 16/333,407

(22) PCT Filed: Sep. 22, 2017
(86) PCT No.: PCT/AU2017/051038

§ 371 Date: Mar. 14, 2019

(30) Foreign Application Priority Data
Sep. 23, 2016 (AU) 2016903858
Jun. 20, 2017 (AU) 2017902352

(51) Int. Cl. C07K 16/36; A61P 7/02

(52) U.S. Cl. CPC C07K 16/36; C07K 2317/76; C07K 2317/31; A61P 7/02

(57) ABSTRACT

A membrane targeted binding protein that binds to at least one blood coagulation factor, wherein the binding protein has pro-coagulant activity.

### RELATED APPLICATION DATA

The present application claims priority from Australian Patent Application No. 2016903858 entitled “Coagulation factor binding proteins and uses thereof” filed on 23 Sep. 2016 and Australian Patent Application No. 2017902352 entitled “Coagulation factor binding proteins and uses thereof” filed on 20 Jun. 2017, the entire contents of which are hereby incorporated by reference.

### SEQUENCE LISTING

The present application is filed with a Sequence Listing in electronic form. The entire contents of the Sequence Listing are hereby incorporated by reference.

### FIELD

The present disclosure relates to coagulation factor binding proteins and uses thereof.

### BACKGROUND

Normal blood coagulation is a highly conserved process in mammalian biology involving complex physiological and biochemical processes comprising activation of a coagulation factor (or clotting factor) cascade ultimately leading to fibrin formation and platelet aggregation. The blood coagulation cascade is comprised of an “extrinsic” pathway, the primary means of coagulation initiation, and an “intrinsic” pathway which contributes to stabilisation of the fibrin clot.

The majority of coagulation factors involved in the coagulation cascade are precursors of proteolytic enzymes known as zymogens. These enzymes circulate in the blood in a non-activated form and only participate in the coagulation cascade once they become activated (e.g. by proteolytic cleavage).

Blood coagulation is inadequate in bleeding disorders, which may be caused by congenital coagulation disorders, acquired coagulation disorders, or haemorrhagic conditions induced by trauma. Congenital coagulation disorders include haemophilia, a recessive X-linked disorder involving a deficiency of coagulation factor VIII (hemophilia A) or factor IX (hemophilia B), and von Willebrand disease, a bleeding disorder involving a severe deficiency of von Willebrand factor.

Acquired coagulation disorders may arise in individuals without a previous history of bleeding as a result of a disease process. For example, acquired coagulation disorders may be caused by inhibitors or autoimmunity against blood coagulation factors, such as factor VIII, von Willebrand factor, factors IX, V, XI, XII and XIII; or by hemostatic disorders, for example caused by liver disease, which may be associated with decreased synthesis of coagulation factors.

Bleeding disorders and coagulation factor deficiencies are typically treated by factor replacement, which is expensive and not always effective. For example, patients receiving chronic factor replacement therapy may produce neutralizing antibodies (i.e. inhibitors) to replacement factors rendering the therapy ineffective. Another disadvantage is the short half-life of the infused coagulation factors resulting in the need for multiple and frequent infusions. Various technologies are being developed for prolonging the half-life of coagulation factors and reducing immunogenicity, including modification by albumin fusion, Fc fusion, PEGylation and sialyation. An alternative approach to the use of recombinant coagulation factors or modified forms thereof has been the generation of antibody-based therapies against one or more coagulation factors in the coagulation cascade, e.g., anti-factor IX or anti-factor IX/X antibodies. Another approach to increase hemostatic efficacy has been to target inhibitors of coagulation, such as tissue factor pathway inhibitor (TFPI). Despite these efforts, the prolongation of coagulation factor half-life remains short and continued repeated treatment is required to prevent the disease.

Thus, there is a need in the art for improving the treatment of bleeding disorders.

### SUMMARY

The present disclosure is based on the inventors' identification that targeting a coagulation factor binding protein to a cellular membrane improves its activity. Membrane targeted binding proteins that bind to at least one blood coagulation factor are capable of pro-coagulant activity.

The findings by the inventors provide the basis for a membrane targeted binding protein that binds to at least one blood coagulation factor. The findings by the inventors also provide the basis for methods for treating a bleeding disorder in a subject.

A pro-coagulant membrane-targeted binding protein of the invention, e.g. a membrane-targeted anti-FIX antibody, is specifically targeted to the site of injury in a subject (e.g. the site of bleeding in a haemophilia patient) and therefore is less likely to cause unwanted coagulation or thrombosis at a site removed or remote from the site of injury, compared to a non-membrane-targeted pro-coagulant protein such as an anti-FIX/FX bispecific antibody.

Accordingly, a pro-coagulant membrane-targeted binding protein of the invention may represent a safer treatment option for patients, e.g. haemophilia patients with FVIII inhibitors receiving a protein with pro-coagulant activity and additional bleeding control (e.g. an activated thrombin complex), than a non-membrane targeted pro-coagulant protein. Targeting the pro-coagulant protein to the site of injury could also permit lower dosing, which would also contribute to a better safety profile compared to a non-membrane-targeted pro-coagulant protein.

For example, the present disclosure provides a membrane targeted binding protein that binds to at least one blood coagulation factor, wherein the binding protein modulates coagulation.

For example, the present disclosure provides a membrane targeted binding protein that binds to at least one blood coagulation factor, wherein the binding protein has pro-coagulant activity.

In one example, the present disclosure provides a membrane targeted binding protein that binds to at least one blood coagulation factor, wherein the binding protein has anti-coagulant activity.

In one example, the present disclosure provides a membrane targeted binding protein (e.g., an antibody or antigen binding fragment thereof) that binds or specifically binds to at least one coagulation factor.

In one example, the present disclosure provides a membrane targeted binding protein that binds to at least one blood coagulation factor, wherein the protein comprises a binding region that specifically binds to the at least one blood coagulation factor. In one example, the binding region specifically binds to one blood coagulation factor and/or an activated form thereof.

In one example, the present disclosure provides a membrane targeted binding protein that binds to at least one blood coagulation factor, wherein the protein comprises a binding region that specifically binds to a component of a plasma membrane of a mammalian cell. In one example, the cell is accessible by plasma or is in contact with plasma, e.g., in its native state. In one example, the cell is within a blood vessel. In one example, the cell is within blood, e.g., is a blood cell.

In one example, the present disclosure provides a membrane targeted binding protein that binds to at least one blood coagulation factor, wherein the protein comprises a first binding region that specifically binds to the at least one blood coagulation factor and a second binding region that specifically binds to a component of a plasma membrane of a mammalian cell. In one example, the first binding region that specifically binds to the at least one blood coagulation factor has pro-coagulant activity.

In one example, the membrane targeted binding protein specifically binds to at least one blood coagulation factor. This does not mean that the membrane targeted binding protein of the present disclosure does not bind to other proteins, only that the membrane targeted binding protein (or part thereof) is specific to a blood coagulation factor and does not bind proteins in general. This term also does not exclude e.g., a bispecific antibody or protein comprising binding regions thereof, which can specifically bind to a first blood coagulation factor with one (or more) binding regions and can specifically bind to another coagulation factor or protein with another binding region.

Binding regions contemplated by the present disclosure can take any of a variety of forms including natural proteins or biological proteins. Exemplary binding regions include a nucleic acid (e.g., an aptamer), a polypeptide, a peptide, a small molecule, an antibody or an antigen binding fragment of an antibody.

In one example, the first and/or second binding region is protein-based, e.g., a peptide, polypeptide or protein. In one example, the first binding region is not a coagulation factor.

In one example, the first and/or second binding region is an antibody mimetic. For example, the first and/or second binding region is a protein comprising an antigen binding domain of an immunoglobulin, e.g., an IgNAR, a camelid antibody or a T cell receptor.

In one example, the first and/or second binding region is a domain antibody (e.g., comprising only a heavy chain variable region or only a light chain variable region) or a heavy chain only antibody (e.g., a camelid antibody or IgNAR) or variable region thereof.

In one example, the first and/or second binding region is a protein comprising a variable region fragment (Fv). For example, the first and/or second binding region is selected from the group consisting of:
(i) a single chain Fv fragment (scFv);
(ii) a dimeric scFv (di-scFv); or
(iii) a diabody;
(iv) a triabody;
(v) a tetrabody;
(vi) a Fab;
(vii) a F(ab′)2;
(viii) a Fv; or
(ix) one of (i) to (viii) linked to a constant region of an antibody, Fc or a heavy chain constant domain (C_{H}) 2 and/or C_{H}3 (optionally, such a protein includes a linker between the Fv and the constant region, Fc or heavy chain constant domain, e.g., a linker as described herein, such as a flexible linker).

In another example, the first and/or second binding region is an antibody. Exemplary antibodies are full-length and/or naked (e.g., unconjugated) antibodies. In one example, an antibody of the present disclosure is a full length antibody.

In one example, the antibody is an IgG or an IgE or an IgM or an IgD or an IgA or an IgY antibody. For example, the antibody is an IgG antibody.

In one example, the IgG antibody is an IgG₁ or an IgG₂ or an IgG₃ or an IgG₄. For example, the antibody is an IgG₁ antibody. In another example, the antibody is an IgG₄ antibody. In one example, the antibody is a stabilized IgG₄ antibody.

In one example, the first and/or second binding region is a protein that is recombinant, chimeric, CDR grafted, humanized, synhumanized, primatized, deimmunized or human.

In one example, the first binding region is monospecific, bispecific, or multispecific. For example, the first binding region is monospecific. In one example, the first binding region is multispecific, for example, the first binding region is bispecific.

In one example, the first binding region is monospecific.

In one example, the first binding region is not bispecific.

In one example, the blood coagulation factor of the present disclosure is selected from the group consisting of factor I, factor II, factor III, factor V, factor VII, factor VIII, factor IX, factor X, factor XI, factor XII factor XIII and an activated form of any of the foregoing.

In one example, the first binding region specifically binds to factor IX and/or factor IXa. In another example, the first binding region specifically binds to factor X and/or factor Xa. In a further example, the first binding region specifically binds to factor IX/IXa and factor X/Xa.

In one example, the present disclosure provides a membrane targeted binding protein which comprises a first binding region that specifically binds to a blood coagulation factor. In one example, the first binding region is an anti-factor IX antibody or antigen binding fragment thereof. In one example, the anti-factor IX antibody or antigen binding fragment thereof binds to non-activated factor IX and/or activated factor IXa. In one example, the anti-factor IX antibody or antigen binding fragment thereof binds to factor IX and/or factor IXa and enhances the activity of factor IX and/or factor IXa. For example, the anti-factor IX antibody or antigen binding fragment thereof binds to factor IXa and enhances the activity of factor IXa. In another example, the anti-factor IX antibody binds to factor IX and enhances factor IX activation. Methods for determining the activity of factor IX and/or factor IXa are known in the art and/or described herein.

In one example, the membrane targeted binding protein comprises a first binding region that has bypassing activity. For example, the binding region substitutes for an endogenous coagulation factor in the coagulation cascade, e.g., the extrinsic coagulation cascade. Thus, the protein can induce coagulation in the absence of the coagulation factor that it bypasses and/or in the presence of inhibitors of the coagulation factor that it bypasses. In one example, the membrane targeted binding protein comprises a first binding region that binds factor IX and does not require activated factor VIIIa for activity to induce coagulation (i.e., the protein bypasses factor VIII/VIIIa).

In one example, the membrane targeted binding protein has increased factor VIII bypassing activity compared to a non-membrane targeted form of the binding protein. For example, the factor VIII bypassing activity of a membrane targeted binding protein of the present disclosure is increased by at least 2 fold, such as about 2.5 fold, or about 3 fold, or about 3.5 fold, or about 4 fold, or about 5 fold, or about 6 fold or about 8 fold or about 10 fold compared to a non-membrane targeted form of the binding protein.

In one example, the first binding region binds to an activated coagulation factor (e.g. activated FIX) and stabilizes the factor in its active conformation.

In one example, the membrane targeted binding protein has a maximal effective concentration (EC₅₀) in an activated partial thromboplastin time (aPTT) assay that is less than a non-membrane targeted form of the binding protein. For example, the EC₅₀ of a membrane targeted binding protein of the present disclosure in an aPTT assay is less than 5 nM, such as about 4.5 nM or about 4 nM or about 3.5 nM or about 3 nM. For example, the EC₅₀ of a membrane targeted binding protein in an aPTT assay is about 2.5 nM or about 2 nM or about 1.5 nM or about 1 nM or about 0.5 nM or about 0.1 nM or about 0.05 nM or about 0.01 nM.

In one example, the antigen binding fragment of the present disclosure is a half antibody. For example, the anti-factor IX antibody is a half antibody comprising a single heavy chain and a single light chain.

In one example, the antigen binding fragment of the present disclosure comprises an IgG₄ constant region or a stabilised IgG₄ constant region.

In one example, the anti-factor IX antibody or antigen binding fragment thereof comprises IgG₄ constant regions or stabilized IgG₄ constant regions. For example, the stabilized IgG₄ constant regions comprise a proline at position 241 of the hinge region according to the system of Kabat (Kabat et al., Sequences of Proteins of Immunological Interest Washington D.C. United States Department of Health and Human Services, 1987 and/or 1991) or a proline at position 228 of the hinge region according to the EU numbering system (Edelman, G. M. et al., Proc. Natl. Acad. USA, 63, 78-85 (1969)).

In one example, the IgG₄ Fc comprises a sequence set forth in any one of SEQ ID NO: 15 to 19.

Exemplary IgG₄ Fc amino acid substitutions include S228P, or S228P and T366W, or S228P, T366S, L368A and Y407V, or T350V, T366L, K392L and T394W, or T350V, L351Y, F405A and Y407V, according to the EU numbering system. In one example, the IgG₄ Fc comprises a sequence set forth in SEQ ID NO: 15.

For example, the human IgG₄ Fc comprises a S228P mutation.

In one example, the IgG₄ Fc comprises a sequence set forth in SEQ ID NO: 16. For example, the human IgG₄ Fc comprises a S228P and T366W mutation.

In one example, the IgG₄ Fc comprises a sequence set forth in SEQ ID NO: 17. For example, the human IgG₄ Fc comprises a S228P, T366S, L368A and Y407V mutation.

In one example, the IgG₄ Fc comprises a sequence set forth in SEQ ID NO: 18. For example, the human IgG₄ Fc comprises a T350V, T366L, K392L and T394W mutation.

In one example, the IgG₄ Fc comprises a sequence set forth in SEQ ID NO: 19. For example, the human IgG₄ Fc comprises a T350V, L351Y, F405A and Y407V mutation.

In one example, the first binding region of the membrane targeted binding protein of the disclosure comprises a V_{H} comprising a sequence set forth in SEQ ID NO: 13 and a V_{L} comprising a sequence set forth in SEQ ID NO: 11.

In one example, the first binding region of the membrane targeted binding protein of the disclosure is an antibody comprising a V_{H} comprising a sequence set forth in SEQ ID NO: 13 and a V_{L} comprising a sequence set forth in SEQ ID NO: 11.

In one example, the first binding region of the membrane targeted binding protein is a half antibody comprising a V_{H} comprising a sequence set forth in SEQ ID NO: 13 and a V_{L} comprising a sequence set forth in SEQ ID NO: 11.

In one example, the first binding region of the membrane targeted binding protein of the disclosure comprises a V_{H} comprising a sequence set forth in SEQ ID NO: 35 and a V_{L} comprising a sequence set forth in SEQ ID NO: 11.

In one example, the first binding region of the membrane targeted binding protein of the disclosure comprises a V_{H} comprising a sequence set forth in SEQ ID NO: 38 and a V_{L} comprising a sequence set forth in SEQ ID NO: 11.

In one example, the first binding region of the membrane targeted binding protein of the disclosure comprises a V_{H} comprising a sequence set forth in SEQ ID NO: 41 and a V_{L} comprising a sequence set forth in SEQ ID NO: 11.

In one example, the first binding region of the membrane targeted binding protein of the disclosure comprises a V_{H} comprising a sequence set forth in SEQ ID NO: 50 and a V_{L} comprising a sequence set forth in SEQ ID NO: 11.

In one example, the amino acid sequence of V_{H} comprises a Tyrosine (T), Isoleucine (I) or Lysine (K) or Glutamic Acid (E) at position 103 and/or Lysine (K) or Tyrosine (Y) at position 104 and/or Proline (P), Threonine (T) or Glycine (G) at position 105 and/or Tryptophan (W) or Glycine (G) at position 106 and/or Glycine (G) or Histidine (H) at position 107 and/or Tyrosine (Y) or Phenylalanine (F) or Tryptophan (W) at position 108.

In one example, the amino acid sequence of V_{H} comprises a Glutamic Acid (E) at position 103, Tyrosine (Y) at position 104, Glycine (G) at position 105, Glycine (G) at position 106, Glycine (G) at position 107 and Tryptophan (W) at position 108.

In one example, the amino acid sequence of V_{H} comprises Tyrosine (T) at position 103, Lysine (K) at position 104, Proline (P) at position 105, Tryptophan (W) at position 106, Glycine (G) at position 107 and Tyrosine (Y) at position 108. In one example, the amino acid sequence of V_{H} comprises Isoleucine (I) at position 103, Lysine (K) at position 104, Threonine (T) at position 105, Tryptophan (W) at position 106, Glycine (G) at position 107 and Tyrosine (Y) at position 108.

In one example, the amino acid sequence of V_{H} comprises Lysine (K) at position 103, Lysine (K) at position 104, Glycine (G) at position 105, Tryptophan (W) at position 106, Histidine (H) at position 107 and Phenylalanine (F) at position 108.

In one example, the first binding region of the membrane targeted binding protein of the disclosure is an antibody comprising a V_{H} comprising a sequence set forth in SEQ ID NO: 35 and a V_{L} comprising a sequence set forth in SEQ ID NO: 11.

In one example, the first binding region of the membrane targeted binding protein of the disclosure is an antibody comprising a V_{H} comprising a sequence set forth in SEQ ID NO: 38 and a V_{L} comprising a sequence set forth in SEQ ID NO: 11.

In one example, the first binding region of the membrane targeted binding protein of the disclosure is an antibody comprising a V_{H} comprising a sequence set forth in SEQ ID NO: 41 and a V_{L} comprising a sequence set forth in SEQ ID NO: 11.

In one example, the first binding region of the membrane targeted binding protein of the disclosure is an antibody comprising a V_{H} comprising a sequence set forth in SEQ ID NO: 50 and a V_{L} comprising a sequence set forth in SEQ ID NO: 11.

In one example, the amino acid sequence of V_{H} comprises a Tyrosine (T), Isoleucine (I) or Lysine (K) or Glutamic Acid (E) at position 103 and/or Lysine (K) or Tyrosine (Y) at position 104 and/or Proline (P), Threonine (T) or Glycine (G) at position 105 and/or Tryptophan (W) or Glycine (G) at position 106 and/or Glycine (G) or Histidine (H) at position 107 and/or Tyrosine (Y) or Phenylalanine (F) or Tryptophan (W) at position 108.

In one example, the amino acid sequence of V_{H} comprises a Glutamic Acid (E) at position 103, Tyrosine (Y) at position 104, Glycine (G) at position 105, Glycine (G) at position 106, Glycine (G) at position 107 and Tryptophan (W) at position 108.

In one example, the amino acid sequence of V_{H} comprises Tyrosine (T) at position 103, Lysine (K) at position 104, Proline (P) at position 105, Tryptophan (W) at position 106, Glycine (G) at position 107 and Tyrosine (Y) at position 108.

In one example, the amino acid sequence of V_{H} comprises Isoleucine (I) at position 103, Lysine (K) at position 104, Threonine (T) at position 105, Tryptophan (W) at position 106, Glycine (G) at position 107 and Tyrosine (Y) at position 108.

In one example, the amino acid sequence of V_{H} comprises Lysine (K) at position 103, Lysine (K) at position 104, Glycine (G) at position 105, Tryptophan (W) at position 106, Histidine (H) at position 107 and Phenylalanine (F) at position 108.

In one example, the first binding region of the membrane targeted binding protein of the disclosure is a half antibody comprising a V_{H} comprising a sequence set forth in SEQ ID NO: 35 and a V_{L} comprising a sequence set forth in SEQ ID NO: 11.

In one example, the first binding region of the membrane targeted binding protein of the disclosure is a half antibody comprising a V_{H} comprising a sequence set forth in SEQ ID NO: 38 and a V_{L} comprising a sequence set forth in SEQ ID NO: 11.

In one example, the first binding region of the membrane targeted binding protein of the disclosure is a half antibody comprising a V_{H} comprising a sequence set forth in SEQ ID NO: 41 and a V_{L} comprising a sequence set forth in SEQ ID NO: 11.

In one example, the first binding region of the membrane targeted binding protein of the disclosure is a half antibody comprising a V_{H} comprising a sequence set forth in SEQ ID NO: 50 and a V_{L} comprising a sequence set forth in SEQ ID NO: 11.

In one example, the amino acid sequence of V_{H} comprises a Tyrosine (T), Isoleucine (I) or Lysine (K) or Glutamic Acid (E) at position 103 and/or Lysine (K) or Tyrosine (Y) at position 104 and/or Proline (P), Threonine (T) or Glycine (G) at position 105 and/or Tryptophan (W) or Glycine (G) at position 106 and/or Glycine (G) or Histidine (H) at position 107 and/or Tyrosine (Y) or Phenylalanine (F) or Tryptophan (W) at position 108.

In one example, the amino acid sequence of V_{H} comprises a Glutamic Acid (E) at position 103, Tyrosine (Y) at position 104, Glycine (G) at position 105, Glycine (G) at position 106, Glycine (G) at position 107 and Tryptophan (W) at position 108.

In one example, the amino acid sequence of V_{H} comprises Tyrosine (T) at position 103, Lysine (K) at position 104, Proline (P) at position 105, Tryptophan (W) at position 106, Glycine (G) at position 107 and Tyrosine (Y) at position 108.

In one example, the amino acid sequence of V_{H} comprises Isoleucine (I) at position 103, Lysine (K) at position 104, Threonine (T) at position 105, Tryptophan (W) at position 106, Glycine (G) at position 107 and Tyrosine (Y) at position 108.

In one example, the amino acid sequence of V_{H} comprises Lysine (K) at position 103, Lysine (K) at position 104, Glycine (G) at position 105, Tryptophan (W) at position 106, Histidine (H) at position 107 and Phenylalanine (F) at position 108.

In one example, the first binding region of the membrane targeted binding protein of the present disclosure is any form of a protein or antibody encoded by a nucleic acid encoding any of the foregoing proteins or antibodies.

In one example, the first binding region of the membrane targeted binding protein of the present disclosure comprises a V_{H} comprising a sequence set forth in any one of SEQ ID NOs: 2 to 7, 34, 37 or 40 and a V_{L} comprising a sequence set forth in SEQ ID NO: 1.

In one example, membrane targeted binding protein of the present disclosure comprises:
(i) a V_{L} sequence set forth in SEQ ID NO: 1, a V_{H} sequence set forth in SEQ ID NO: 2 and a V_{H} sequence set forth in SEQ ID NO: 3; or
(ii) a V_{L} sequence set forth in SEQ ID NO: 1 and a V_{H} sequence set forth in SEQ ID NO: 4; or
(iii) a V_{L} sequence set forth in SEQ ID NO: 1 and a V_{H} sequence set forth in SEQ ID NO: 5; or
(iv) a V_{L} sequence set forth in SEQ ID NO: 1 and a V_{H} sequence set forth in SEQ ID NO: 6; or
(v) a V_{L} sequence set forth in SEQ ID NO: 1 and a V_{H} sequence set forth in SEQ ID NO: 7; or
(vi) a V_{L} sequence set forth in SEQ ID NO: 1 and a V_{H} sequence set forth in SEQ ID NO: 8; or
(vii) a V_{L} sequence set forth in SEQ ID NO: 1 and a V_{H} sequence set forth in SEQ ID NO: 9; or
(viii) a V_{L} sequence set forth in SEQ ID NO: 1 and a V_{H} sequence set forth in SEQ ID NO: 10; or
(ix) a V_{L} sequence set forth in SEQ ID NO: 1, a V_{H} sequence set forth in SEQ ID NO: 32; or
(x) a V_{L} sequence set forth in SEQ ID NO: 1, a V_{H} sequence set forth in SEQ ID NO: 33; or
(xi) a V_{L} sequence set forth in SEQ ID NO: 1, a V_{H} sequence set forth in SEQ ID NO: 9 and a V_{H} sequence set forth in SEQ ID NO: 10; or
(xii) a V_{L} sequence set forth in SEQ ID NO: 1, a V_{H} sequence set forth in SEQ ID NO: 6 and a V_{H} sequence set forth in SEQ ID NO: 10; or
(xiii) a V_{L} sequence set forth in SEQ ID NO: 1, a V_{H} sequence set forth in SEQ ID NO: 9 and a V_{H} sequence set forth in SEQ ID NO: 7.

In one example, membrane targeted binding protein of the present disclosure comprises:
(i) a V_{L} sequence set forth in SEQ ID NO: 1, a V_{H} sequence set forth in SEQ ID NO: 2 and a V_{H} sequence set forth in SEQ ID NO: 3; or
(ii) a V_{L} sequence set forth in SEQ ID NO: 1 and a V_{H} sequence set forth in SEQ ID NO: 4; or
(iii) a V_{L} sequence set forth in SEQ ID NO: 1 and a V_{H} sequence set forth in SEQ ID NO: 5; or
(iv) a V_{L} sequence set forth in SEQ ID NO: 1 and a V_{H} sequence set forth in SEQ ID NO: 6; or
(v) a V_{L} sequence set forth in SEQ ID NO: 1 and a V_{H} sequence set forth in SEQ ID NO: 7; or
(vi) a V_{L} sequence set forth in SEQ ID NO: 1 and a V_{H} sequence set forth in SEQ ID NO: 8; or
(vii) a V_{L} sequence set forth in SEQ ID NO: 1 and a V_{H} sequence set forth in SEQ ID NO: 9; or
(viii) a V_{L} sequence set forth in SEQ ID NO: 1 and a V_{H} sequence set forth in SEQ ID NO: 10; or
(ix) a V_{L} sequence set forth in SEQ ID NO: 1, a V_{H} sequence set forth in SEQ ID NO: 31; or
(x) a V_{L} sequence set forth in SEQ ID NO: 1, a V_{H} sequence set forth in SEQ ID NO: 32; or
(xi) a V_{L} sequence set forth in SEQ ID NO: 1, a V_{H} sequence set forth in SEQ ID NO: 33; or
(xii) a V_{L} sequence set forth in SEQ ID NO: 1, a V_{H} sequence set forth in SEQ ID NO: 34; or
(xiii) a V_{L} sequence set forth in SEQ ID NO: 1, a V_{H} sequence set forth in SEQ ID NO: 36; or
(xiv) a V_{L} sequence set forth in SEQ ID NO: 1, a V_{H} sequence set forth in SEQ ID NO: 37; or
(xv) a V_{L} sequence set forth in SEQ ID NO: 1, a V_{H} sequence set forth in SEQ ID NO: 39; or
(xvi) a V_{L} sequence set forth in SEQ ID NO: 1, a V_{H} sequence set forth in SEQ ID NO: 40; or
(xvii) a V_{L} sequence set forth in SEQ ID NO: 1, a V_{H} sequence set forth in SEQ ID NO: 42; or
(xviii) a V_{L} sequence set forth in SEQ ID NO: 1, a V_{H} sequence set forth in SEQ ID NO: 9 and a V_{H} sequence set forth in SEQ ID NO: 10; or
(xix) a V_{L} sequence set forth in SEQ ID NO: 1, a V_{H} sequence set forth in SEQ ID NO: 6 and a V_{H} sequence set forth in SEQ ID NO: 10; or
(xx) a V_{L} sequence set forth in SEQ ID NO: 1, a V_{H} sequence set forth in SEQ ID NO: 9 and a V_{H} sequence set forth in SEQ ID NO: 7; or
(xxi) a V_{L} sequence set forth in SEQ ID NO: 1, a V_{H} sequence set forth in SEQ ID NO: 54; or
(xxii) a V_{L} sequence set forth in SEQ ID NO: 1, a V_{H} sequence set forth in SEQ ID NO: 55.

In one example, the V_{H} CDR1 comprises amino acids 31 to 35 of SEQ ID NO:13, the V_{H} CDR2 comprises amino acids 50 to 59 of SEQ ID NO:13 and the V_{H} CDR3 comprises amino acids 99 to 106 of SEQ ID NO:13.

In one example, the V_{L} CDR1 comprises amino acids 24 to 34 of SEQ ID NO: 11; the V_{L} CDR2 comprises amino acids 50 to 56 of SEQ ID NO: 11; and the V_{L} CDR3 comprises amino acids 89 to 97 of SEQ ID NO: 11.

In one example, the first binding region of the membrane targeted binding protein comprises:
(i) a V_{H} comprising:
   (a) a CDR1 comprising a sequence set forth in amino acids 31 to 35 of SEQ ID NO: 13;
   (b) a CDR2 comprising a sequence set forth in amino acids 50 to 66 of SEQ ID NO: 13; and
   (c) a CDR3 comprising a sequence set forth in amino acids 99 to 112 of SEQ ID NO: 13; and
(ii) a V_{L} comprising:
   (a) a CDR1 comprising a sequence set forth in amino acids 24 to 34 of SEQ ID NO: 11;
   (b) a CDR2 comprising a sequence set forth in amino acids 50 to 56 of SEQ ID NO: 11; and
   (c) a CDR3 comprising a sequence set forth in amino acids 89 to 97 of SEQ ID NO: 11.

In one example, the first binding region of the membrane targeted binding protein is an antibody comprising:
(i) a V_{H} comprising:
   (a) a CDR1 comprising a sequence set forth in amino acids 31 to 35 of SEQ ID NO: 13;
   (b) a CDR2 comprising a sequence set forth in amino acids 50 to 66 of SEQ ID NO: 13; and
   (c) a CDR3 comprising a sequence set forth in amino acids 99 to 112 of SEQ ID NO: 13; and
(ii) a V_{L} comprising:
   (a) a CDR1 comprising a sequence set forth in amino acids 24 to 34 of SEQ ID NO: 11;
   (b) a CDR2 comprising a sequence set forth in amino acids 50 to 56 of SEQ ID NO: 11; and
   (c) a CDR3 comprising a sequence set forth in amino acids 89 to 97 of SEQ ID NO: 11.

In one example, the first binding region of the membrane targeted binding protein is a half antibody comprising:
(i) a V_{H} comprising:
   (a) a CDR1 comprising a sequence set forth in amino acids 31 to 35 of SEQ ID NO: 13;
   (b) a CDR2 comprising a sequence set forth in amino acids 50 to 66 of SEQ ID NO: 13; and
   (c) a CDR3 comprising a sequence set forth in amino acids 99 to 112 of SEQ ID NO: 13; and
(ii) a V_{L} comprising:
   (a) a CDR1 comprising a sequence set forth in amino acids 24 to 34 of SEQ ID NO: 11;
   (b) a CDR2 comprising a sequence set forth in amino acids 50 to 56 of SEQ ID NO: 11; and
   (c) a CDR3 comprising a sequence set forth in amino acids 89 to 97 of SEQ ID NO: 11.

In one example, the V_{H} CDR1 comprises the amino acid sequence shown in SEQ ID NO:43, the V_{H} CDR2 comprises the amino acid sequence shown in SEQ ID NO:44 and the V_{H} CDR3 comprises the amino acid sequence shown in SEQ ID NO:45.

In one example, the V_{L} CDR1 comprises the amino acid sequence shown in SEQ ID NO: 47, the V_{L} CDR2 comprises the amino acid sequence shown in SEQ ID NO: 48 and the V_{L} CDR3 comprises the amino acid sequence shown in SEQ ID NO: 49. In one example, the first binding region of the membrane targeted binding protein comprises:
(i) a V_{H} comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 45; and
(ii) a V_{L} comprising:
   (a) a CDR1 comprising a sequence set forth in SEQ ID NO: 47;
   (b) a CDR2 comprising a sequence set forth in SEQ ID NO: 48; and
   (c) a CDR3 comprising a sequence set forth in SEQ ID NO: 49.

In one example, the first binding region of the membrane targeted binding protein is an antibody comprising:
(i) a V_{H} comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 45; and
(ii) a V_{L} comprising:
   (a) a CDR1 comprising a sequence set forth in SEQ ID NO: 47;
   (b) a CDR2 comprising a sequence set forth in SEQ ID NO: 48; and
   (c) a CDR3 comprising a sequence set forth in SEQ ID NO: 49.

In one example, the first binding region of the membrane targeted binding protein is a half antibody comprising:
(i) a V_{H} comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 45; and
(ii) a V_{L} comprising:
   (a) a CDR1 comprising a sequence set forth in SEQ ID NO: 47;
   (b) a CDR2 comprising a sequence set forth in SEQ ID NO: 48; and
   (c) a CDR3 comprising a sequence set forth in SEQ ID NO: 49.

In one example, the V_{H} CDR1 comprises amino acids 31 to 35 of any one of SEQ ID NOs: 13, 35, 38 or 41, the V_{H} CDR2 comprises amino acids 50 to 59 of any one of SEQ ID NOs: 13, 35, 38 or 41 and the V_{H} CDR3 comprises amino acids 99 to 106 of any one of SEQ ID NOs: 35, 38, 41 or 50.

In one example, the first binding region of the membrane targeted binding protein comprises:
(i) a V_{H} comprising:
   (a) a CDR1 comprising a sequence set forth in amino acids 31 to 35 of SEQ ID NO: 35;
   (b) a CDR2 comprising a sequence set forth in amino acids 50 to 66 of SEQ ID NO: 35; and
   (c) a CDR3 comprising a sequence set forth in amino acids 99 to 112 of SEQ ID NO: 35; and
(ii) a V_{L} comprising:
   (a) a CDR1 comprising a sequence set forth in amino acids 24 to 34 of SEQ ID NO: 11;
   (b) a CDR2 comprising a sequence set forth in amino acids 50 to 56 of SEQ ID NO: 11; and
   (c) a CDR3 comprising a sequence set forth in amino acids 89 to 97 of SEQ ID NO: 11.

In one example, the first binding region of the membrane targeted binding protein is an antibody comprising:
(i) a V_{H} comprising:
   (a) a CDR1 comprising a sequence set forth in amino acids 31 to 35 of SEQ ID NO: 35;
   (b) a CDR2 comprising a sequence set forth in amino acids 50 to 66 of SEQ ID NO: 35; and
   (c) a CDR3 comprising a sequence set forth in amino acids 99 to 112 of SEQ ID NO: 35; and
(ii) a V_{L} comprising:
   (a) a CDR1 comprising a sequence set forth in amino acids 24 to 34 of SEQ ID NO: 11;
   (b) a CDR2 comprising a sequence set forth in amino acids 50 to 56 of SEQ ID NO: 11; and
   (c) a CDR3 comprising a sequence set forth in amino acids 89 to 97 of SEQ ID NO: 11.

In one example, the first binding region of the membrane targeted binding protein is a half antibody comprising:
(i) a V_{H} comprising:
   (a) a CDR1 comprising a sequence set forth in amino acids 31 to 35 of SEQ ID NO: 35;
   (b) a CDR2 comprising a sequence set forth in amino acids 50 to 66 of SEQ ID NO: 35; and
   (c) a CDR3 comprising a sequence set forth in amino acids 99 to 112 of SEQ ID NO: 35; and
(ii) a V_{L} comprising:
   (a) a CDR1 comprising a sequence set forth in amino acids 24 to 34 of SEQ ID NO: 11;
   (b) a CDR2 comprising a sequence set forth in amino acids 50 to 56 of SEQ ID NO: 11; and
   (c) a CDR3 comprising a sequence set forth in amino acids 89 to 97 of SEQ ID NO: 11.

In one example, the first binding region of the membrane targeted binding protein comprises:
(i) a V_{H} comprising:
   (a) a CDR1 comprising a sequence set forth in amino acids 31 to 35 of SEQ ID NO: 38;
   (b) a CDR2 comprising a sequence set forth in amino acids 50 to 66 of SEQ ID NO: 38; and
   (c) a CDR3 comprising a sequence set forth in amino acids 99 to 112 of SEQ ID NO: 38; and
(ii) a V_{L} comprising:
   (a) a CDR1 comprising a sequence set forth in amino acids 24 to 34 of SEQ ID NO: 11;
   (b) a CDR2 comprising a sequence set forth in amino acids 50 to 56 of SEQ ID NO: 11; and
   (c) a CDR3 comprising a sequence set forth in amino acids 89 to 97 of SEQ ID NO: 11.

In one example, the first binding region of the membrane targeted binding protein is an antibody comprising:
(i) a V_{H} comprising:
   (a) a CDR1 comprising a sequence set forth in amino acids 31 to 35 of SEQ ID NO: 38;
   (b) a CDR2 comprising a sequence set forth in amino acids 50 to 66 of SEQ ID NO: 38; and
   (c) a CDR3 comprising a sequence set forth in amino acids 99 to 112 of SEQ ID NO: 38; and
(ii) a V_{L} comprising:
   (a) a CDR1 comprising a sequence set forth in amino acids 24 to 34 of SEQ ID NO: 11;
   (b) a CDR2 comprising a sequence set forth in amino acids 50 to 56 of SEQ ID NO: 11; and
   (c) a CDR3 comprising a sequence set forth in amino acids 89 to 97 of SEQ ID NO: 11.

In one example, the first binding region of the membrane targeted binding protein is a half antibody comprising:
(i) a V_{H} comprising:
   (a) a CDR1 comprising a sequence set forth in amino acids 31 to 35 of SEQ ID NO: 38;
   (b) a CDR2 comprising a sequence set forth in amino acids 50 to 66 of SEQ ID NO: 38; and
   (c) a CDR3 comprising a sequence set forth in amino acids 99 to 112 of SEQ ID NO: 38; and
(ii) a V_{L} comprising:
   (a) a CDR1 comprising a sequence set forth in amino acids 24 to 34 of SEQ ID NO: 11;
   (b) a CDR2 comprising a sequence set forth in amino acids 50 to 56 of SEQ ID NO: 11; and
   (c) a CDR3 comprising a sequence set forth in amino acids 89 to 97 of SEQ ID NO: 11.

In one example, the first binding region of the membrane targeted binding protein comprises:
(i) a V_{H} comprising:
   (a) a CDR1 comprising a sequence set forth in amino acids 31 to 35 of SEQ ID NO: 41;
   (b) a CDR2 comprising a sequence set forth in amino acids 50 to 66 of SEQ ID NO: 41; and
   (c) a CDR3 comprising a sequence set forth in amino acids 99 to 112 of SEQ ID NO: 41; and
(ii) a V_{L} comprising:
   (a) a CDR1 comprising a sequence set forth in amino acids 24 to 34 of SEQ ID NO: 11;
   (b) a CDR2 comprising a sequence set forth in amino acids 50 to 56 of SEQ ID NO: 11; and
   (c) a CDR3 comprising a sequence set forth in amino acids 89 to 97 of SEQ ID NO: 11.

In one example, the first binding region of the membrane targeted binding protein is an antibody comprising:
(i) a V_{H} comprising:
   (a) a CDR1 comprising a sequence set forth in amino acids 31 to 35 of SEQ ID NO: 41;
   (b) a CDR2 comprising a sequence set forth in amino acids 50 to 66 of SEQ ID NO: 41; and
   (c) a CDR3 comprising a sequence set forth in amino acids 99 to 112 of SEQ ID NO: 41; and
(ii) a V_{L} comprising:
   (a) a CDR1 comprising a sequence set forth in amino acids 24 to 34 of SEQ ID NO: 11;
   (b) a CDR2 comprising a sequence set forth in amino acids 50 to 56 of SEQ ID NO: 11; and
   (c) a CDR3 comprising a sequence set forth in amino acids 89 to 97 of SEQ ID NO: 11.

In one example, the first binding region of the membrane targeted binding protein is a half antibody comprising:
(i) a V_{H} comprising:
   (a) a CDR1 comprising a sequence set forth in amino acids 31 to 35 of SEQ ID NO: 41;
   (b) a CDR2 comprising a sequence set forth in amino acids 50 to 66 of SEQ ID NO: 41; and
   (c) a CDR3 comprising a sequence set forth in amino acids 99 to 112 of SEQ ID NO: 41; and
(ii) a V_{L} comprising:
   (a) a CDR1 comprising a sequence set forth in amino acids 24 to 34 of SEQ ID NO: 11;
   (b) a CDR2 comprising a sequence set forth in amino acids 50 to 56 of SEQ ID NO: 11; and
   (c) a CDR3 comprising a sequence set forth in amino acids 89 to 97 of SEQ ID NO: 11.

In one example, the first binding region of the membrane targeted binding protein comprises:
(i) a V_{H} comprising:
   (a) a CDR1 comprising a sequence set forth in amino acids 31 to 35 of any one of SEQ ID NOs: 13, 35, 38 or 41;
   (b) a CDR2 comprising a sequence set forth in amino acids 50 to 66 of any one of SEQ ID NOs: 13, 35, 38 or 41; and
   (c) a CDR3 comprising a sequence set forth in amino acids 99 to 112 of SEQ ID NO: 50; and
(ii) a V_{L} comprising:
   (a) a CDR1 comprising a sequence set forth in amino acids 24 to 34 of SEQ ID NO: 11;
   (b) a CDR2 comprising a sequence set forth in amino acids 50 to 56 of SEQ ID NO: 11; and
   (c) a CDR3 comprising a sequence set forth in amino acids 89 to 97 of SEQ ID NO: 11.

In one example, the first binding region of the membrane targeted binding protein is an antibody comprising:
(i) a V_{H} comprising:
   (a) a CDR1 comprising a sequence set forth in amino acids 31 to 35 of any one of SEQ ID NOs: 13, 35, 38 or 41;
   (b) a CDR2 comprising a sequence set forth in amino acids 50 to 66 of any one of SEQ ID NOs: 13, 35, 38 or 41; and
   (c) a CDR3 comprising a sequence set forth in amino acids 99 to 112 of SEQ ID NO: 50; and
(ii) a V_{L} comprising:
   (a) a CDR1 comprising a sequence set forth in amino acids 24 to 34 of SEQ ID NO: 11;
   (b) a CDR2 comprising a sequence set forth in amino acids 50 to 56 of SEQ ID NO: 11; and
   (c) a CDR3 comprising a sequence set forth in amino acids 89 to 97 of SEQ ID NO: 11.

In one example, the first binding region of the membrane targeted binding protein is a half antibody comprising:
(i) a V_{H} comprising:
   (a) a CDR1 comprising a sequence set forth in amino acids 31 to 35 of any one of SEQ ID NOs: 13, 35, 38 or 41;
   (b) a CDR2 comprising a sequence set forth in amino acids 50 to 66 of any one of SEQ ID NOs: 13, 35, 38 or 41; and
   (c) a CDR3 comprising a sequence set forth in amino acids 99 to 112 of SEQ ID NO: 50; and
(ii) a V_{L} comprising:
   (a) a CDR1 comprising a sequence set forth in amino acids 24 to 34 of SEQ ID NO: 11;
   (b) a CDR2 comprising a sequence set forth in amino acids 50 to 56 of SEQ ID NO: 11; and
   (c) a CDR3 comprising a sequence set forth in amino acids 89 to 97 of SEQ ID NO: 11.

In one example, the V_{H} CDR1 comprises the amino acid sequence shown in SEQ ID NO: 43, the V_{H} CDR2 comprises the amino acid sequence shown in SEQ ID NO: 44 and the V_{H} CDR3 comprises the amino acid sequence shown in any one of SEQ ID NOs: 46 or 51 to 53.

In one example, the first binding region of the membrane targeted binding protein comprises:
(i) a V_{H} comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 46; and
(ii) a V_{L} comprising:
   (a) a CDR1 comprising a sequence set forth in SEQ ID NO: 47;
   (b) a CDR2 comprising a sequence set forth in SEQ ID NO: 48; and
   (c) a CDR3 comprising a sequence set forth in SEQ ID NO: 49.

In one example, the first binding region of the membrane targeted binding protein is an antibody comprising:
(i) a V_{H} comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 46; and
(ii) a V_{L} comprising:
   (a) a CDR1 comprising a sequence set forth in SEQ ID NO: 47;
   (b) a CDR2 comprising a sequence set forth in SEQ ID NO: 48; and
   (c) a CDR3 comprising a sequence set forth in SEQ ID NO: 49.

In one example, the first binding region of the membrane targeted binding protein is a half antibody comprising:
(i) a V_{H} comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 46; and
(ii) a V_{L} comprising:
   (a) a CDR1 comprising a sequence set forth in SEQ ID NO: 47;
   (b) a CDR2 comprising a sequence set forth in SEQ ID NO: 48; and
   (c) a CDR3 comprising a sequence set forth in SEQ ID NO: 49.

In one example, the first binding region of the membrane targeted binding protein comprises:
(i) a V_{H} comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 51; and
(ii) a V_{L} comprising:
   (a) a CDR1 comprising a sequence set forth in SEQ ID NO: 47;
   (b) a CDR2 comprising a sequence set forth in SEQ ID NO: 48; and
   (c) a CDR3 comprising a sequence set forth in SEQ ID NO: 49.

In one example, the first binding region of the membrane targeted binding protein is an antibody comprising:
(i) a V_{H} comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 51; and
(ii) a V_{L} comprising:
   (a) a CDR1 comprising a sequence set forth in SEQ ID NO: 47;
   (b) a CDR2 comprising a sequence set forth in SEQ ID NO: 48; and
   (c) a CDR3 comprising a sequence set forth in SEQ ID NO: 49.

In one example, the first binding region of the membrane targeted binding protein is a half antibody comprising:
(i) a V_{H} comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 51; and
(ii) a V_{L} comprising:
   (a) a CDR1 comprising a sequence set forth in SEQ ID NO: 47;
   (b) a CDR2 comprising a sequence set forth in SEQ ID NO: 48; and
   (c) a CDR3 comprising a sequence set forth in SEQ ID NO: 49.

In one example, the first binding region of the membrane targeted binding protein comprises:
(i) a V_{H} comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 52; and
(ii) a V_{L} comprising:
   (a) a CDR1 comprising a sequence set forth in SEQ ID NO: 47;
   (b) a CDR2 comprising a sequence set forth in SEQ ID NO: 48; and
   (c) a CDR3 comprising a sequence set forth in SEQ ID NO: 49.

In one example, the first binding region of the membrane targeted binding protein is an antibody comprising:
(i) a V_{H} comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 52; and
(ii) a V_{L} comprising:
   (a) a CDR1 comprising a sequence set forth in SEQ ID NO: 47;
   (b) a CDR2 comprising a sequence set forth in SEQ ID NO: 48; and
   (c) a CDR3 comprising a sequence set forth in SEQ ID NO: 49.

In one example, the first binding region of the membrane targeted binding protein is a half antibody comprising:
(i) a V_{H} comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 52; and
(ii) a V_{L} comprising:
   (a) a CDR1 comprising a sequence set forth in SEQ ID NO: 47;
   (b) a CDR2 comprising a sequence set forth in SEQ ID NO: 48; and
   (c) a CDR3 comprising a sequence set forth in SEQ ID NO: 49.

In one example, the first binding region of the membrane targeted binding protein comprises:
(i) a V_{H} comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 53; and
(ii) a V_{L} comprising:
   (a) a CDR1 comprising a sequence set forth in SEQ ID NO: 47;
   (b) a CDR2 comprising a sequence set forth in SEQ ID NO: 48; and
   (c) a CDR3 comprising a sequence set forth in SEQ ID NO: 49.

In one example, the first binding region of the membrane targeted binding protein is an antibody comprising:
(i) a V_{H} comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 53; and
(ii) a V_{L} comprising:
   (a) a CDR1 comprising a sequence set forth in SEQ ID NO: 47;
   (b) a CDR2 comprising a sequence set forth in SEQ ID NO: 48; and
   (c) a CDR3 comprising a sequence set forth in SEQ ID NO: 49.

In one example, the first binding region of the membrane targeted binding protein is a half antibody comprising:
(i) a V_{H} comprising:
   (a) a CDR1 comprising a sequence set forth in SEQ ID NO: 43;
   (b) a CDR2 comprising a sequence set forth in SEQ ID NO: 44; and
   (c) a CDR3 comprising a sequence set forth in SEQ ID NO: 53; and
(ii) a V_{L} comprising:
   (a) a CDR1 comprising a sequence set forth in SEQ ID NO: 47;
   (b) a CDR2 comprising a sequence set forth in SEQ ID NO: 48; and
   (c) a CDR3 comprising a sequence set forth in SEQ ID NO: 49.

In one example, the V_{H} comprises the amino acid sequence shown in SEQ ID NO: 43, the V_{H} CDR2 comprises the amino acid sequence shown in SEQ ID NO: 44 and the V_{H} CDR2 comprises the amino acid sequence shown in SEQ ID NO: 46.

In one example, the amino acid sequence of V_{H} CDR3 comprises a Tyrosine (T), Isoleucine (I) or Lysine (K) or Glutamic Acid (E) at position 5 and/or Lysine (K) or Tyrosine (Y) at position 6 and/or Proline (P), Threonine (T) or Glycine (G) at position 7 and/or Tryptophan (W) or Glycine (G) at position 8 and/or Glycine (G) or Histidine (H) at position 9 and/or Tyrosine (Y) or Phenylalanine (F) or Tryptophan (W) at position 10.

In one example, the amino acid sequence of V_{H} CDR3 comprises a Glutamic Acid (E) at position 5, Tyrosine (Y) at position 6, Glycine (G) at position 7, Glycine (G) at position 8, Glycine (G) at position 9 and Tryptophan (W) at position 10.

In one example, the amino acid sequence of V_{H} CDR3 comprises Tyrosine (T) at position 5, Lysine (K) at position 6, Proline (P) at position 7, Tryptophan (W) at position 8, Glycine (G) at position 9 and Tyrosine (Y) at position 10.

In one example, the amino acid sequence of V_{H} CDR3 comprises Isoleucine (I) at position 5, Lysine (K) at position 6, Threonine (T) at position 7, Tryptophan (W) at position 8, Glycine (G) at position 9 and Tyrosine (Y) at position 10.

In one example, the amino acid sequence of V_{H} CDR3 comprises Lysine (K) at position 5, Lysine (K) at position 6, Glycine (G) at position 7, Tryptophan (W) at position 8, Histidine (H) at position 9 and Phenylalanine (F) at position 10.

In one example, the membrane targeted binding protein comprises a light chain sequence set forth in SEQ ID NO: 1, a heavy chain sequence set forth in SEQ ID NO: 2 and a heavy chain sequence set forth in SEQ ID NO: 3.

In one example, the membrane targeted binding protein comprises a light chain sequence set forth in SEQ ID NO: 1, a heavy chain sequence set forth in SEQ ID NO: 4.

In one example, the membrane targeted binding protein comprises a light chain sequence set forth in SEQ ID NO: 1 and a heavy chain sequence set forth in SEQ ID NO: 5.

In one example, the membrane targeted binding protein comprises a light chain sequence set forth in SEQ ID NO: 1 and a heavy chain sequence set forth in SEQ ID NO: 6.

In one example, the membrane targeted binding protein comprises a light chain sequence set forth in SEQ ID NO: 1, a heavy chain sequence set forth in SEQ ID NO: 7.

In one example, the membrane targeted binding protein comprises a light chain sequence set forth in SEQ ID NO: 1 and a heavy chain sequence set forth in SEQ ID NO: 8.

In one example, the membrane targeted binding protein comprises a light chain sequence set forth in SEQ ID NO: 1 and a heavy chain sequence set forth in SEQ ID NO: 9.

In one example, the membrane targeted binding protein comprises a light chain sequence set forth in SEQ ID NO: 1 and a heavy chain sequence set forth in SEQ ID NO: 10.

In one example, the membrane targeted binding protein comprises a light chain sequence set forth in SEQ ID NO: 1 and a heavy chain sequence set forth in SEQ ID NO: 31.

In one example, the membrane targeted binding protein comprises a light chain sequence set forth in SEQ ID NO: 1 and a heavy chain sequence set forth in SEQ ID NO: 32.

In one example, the membrane targeted binding protein comprises a light chain sequence set forth in SEQ ID NO: 1 and a heavy chain sequence set forth in SEQ ID NO: 33.

In one example, the membrane targeted binding protein comprises a light chain sequence set forth in SEQ ID NO: 1 and a heavy chain sequence set forth in SEQ ID NO: 34.

In one example, the membrane targeted binding protein comprises a light chain sequence set forth in SEQ ID NO: 1 and a heavy chain sequence set forth in SEQ ID NO: 36.

In one example, the membrane targeted binding protein comprises a light chain sequence set forth in SEQ ID NO: 1 and a heavy chain sequence set forth in SEQ ID NO: 37.

In one example, the membrane targeted binding protein comprises a light chain sequence set forth in SEQ ID NO: 1 and a heavy chain sequence set forth in SEQ ID NO: 39.

In one example, the membrane targeted binding protein comprises a light chain sequence set forth in SEQ ID NO: 1 and a heavy chain sequence set forth in SEQ ID NO: 40.

In one example, the membrane targeted binding protein comprises a light chain sequence set forth in SEQ ID NO: 1 and a heavy chain sequence set forth in SEQ ID NO: 42.

In one example, the membrane targeted binding protein comprises a light chain sequence set forth in SEQ ID NO: 1 and a heavy chain sequence set forth in SEQ ID NO: 54.

In one example, the membrane targeted binding protein comprises a light chain sequence set forth in SEQ ID NO: 1 and a heavy chain sequence set forth in SEQ ID NO: 55.

In one example, the membrane targeted binding protein comprises a light chain sequence set forth in SEQ ID NO: 1, a heavy chain sequence set forth in SEQ ID NO: 9 and a heavy chain sequence set forth in SEQ ID NO: 10.

In one example, the membrane targeted binding protein comprises a light chain sequence set forth in SEQ ID NO: 1, a heavy chain sequence set forth in SEQ ID NO: 6 and a heavy chain sequence set forth in SEQ ID NO: 10.

In one example, the membrane targeted binding protein comprises a light chain sequence set forth in SEQ ID NO: 1, a heavy chain sequence set forth in SEQ ID NO: 9 and a heavy chain sequence set forth in SEQ ID NO: 7.

In one example, the membrane targeted binding protein of the present disclosure comprises a second binding region that specifically binds to a component of a plasma membrane of a mammalian cell.

In one example, the second binding region of the membrane targeted binding protein is selected from the group consisting of an antibody or antigen binding fragment thereof, an annexin or a variant thereof, a gamma-carboxyglutamic acid (GLA) domain or a variant thereof, a lactadherin domain or a fragment/variant thereof, a protein kinase C (PKC) domain, a PKC conserved 1 (C1) domain, a PKC conserved 2 (C2) domain a pleckstrin homology domain, and a PSP1 peptide (comprising a sequence set forth in SEQ ID NO: 28) or a variant thereof. For example, the lactadherin fragment is a C1C2 fragment, e.g., as set forth in SEQ ID NO: 27.

In one example, the second binding region of the membrane targeted binding protein is a non-antibody-based protein, e.g., selected from the group consisting of an annexin or a variant thereof, a gamma-carboxyglutamic acid (GLA) domain or a variant thereof, a lactadherin domain or a fragment/variant thereof, a protein kinase C (PKC) domain, a PKC conserved 1 (C1) domain, a PKC conserved 2 (C2) domain a pleckstrin homology domain, and a PSP1 peptide (comprising a sequence set forth in SEQ ID NO: 28) or a variant thereof. For example, the lactadherin fragment is a C1C2 fragment, e.g., as set forth in SEQ ID NO: 27.

In one example, the second binding region of the membrane targeted binding protein is not involved in or does not have procoagulant activity.

In one example, the second binding region of the membrane targeted binding protein is an annexin or a variant thereof, or a phosphatidylserine binding fragment of an annexin or variant thereof. Exemplary variants of annexin are known in the art and/or described therein. In one example, the second binding region is an annexin. For example, the annexin is Annexin A5. In one example, the second binding region of the membrane targeted binding protein is Annexin A5 comprising a sequence set forth in SEQ ID NO: 14. In one example, the second binding region of the membrane targeted binding protein is the E5 mutant of Annexin A5 comprising a sequence set forth in SEQ ID NO: 26 (which corresponds to the Annexin A5 quintuple mutant disclosed in Bouter et al, Nature Communications 2:270 (2011), i.e., comprising R16E, R23E, K27E, K56E and K191E mutations in Annexin A5). In another example, the second binding region of the membrane targeted binding protein is Annexin A1. For example, the second binding region of the membrane targeted binding protein is Annexin A1 comprising a sequence set forth in SEQ ID NO: 29. In another example, the second binding region of the membrane targeted binding protein is a truncated Annexin A1 comprising a sequence set forth in SEQ ID NO: 30 (in which the 41 N-terminal amino acids comprising a self-cleavage site have been deleted from wild-type Annexin A1).

In one example, the membrane targeted binding protein of the present disclosure comprises an antibody wherein each heavy chain of the antibody is linked to an annexin that binds to a component of a plasma membrane of a mammalian cell. For example, each heavy chain of the antibody is linked to an annexin, such as Annexin A5 or Annexin A1. In another example, only one of the heavy chains of the antibody is linked to an annexin.

In one example, the second binding region binds to a component of the plasma membrane selected from the group consisting of an aminophospholipid, a membrane-associated polypeptide and mixtures thereof.

In one example, the component of the plasma membrane is an aminophospholipid. For example, the component of the plasma membrane is an aminophospholipid selected from the group consisting of a phosphatidylserine, a phosphatidylethanolamine and mixtures thereof.

In one example, the component of the plasma membrane is a membrane-associated polypeptide. In one example, the membrane-associated polypeptide is selected from the group consisting of GPIIb/IIIa, β2GP1, TLT-1, a coagulation factor, a selectin and mixtures thereof.

In one example, the mammalian cell is selected from the group consisting of a platelet, an endothelial cell and a red blood cell. For example, the mammalian cell is a platelet.

In one example, the membrane targeted binding protein that binds to at least one blood coagulation factor comprises:
(i) a first binding region comprising an anti-Factor IX antibody or antigen binding fragment thereof; and
(ii) a second binding region comprising:
   (a) Annexin A5 comprising a sequence set forth in SEQ ID NO: 14; or
   (b) truncated Annexin A1 comprising a sequence set forth in SEQ ID NO: 30;
   (c) a phosphatidylserine binding fragment or variant of Annexin A5; or
   (d) a phosphatidylserine binding fragment or variant of Annexin A1.

In one example, the present disclosure provides a membrane targeted binding protein that binds to a coagulation factor, wherein the protein comprises:
(i) a first binding region that is an anti-Factor IX half antibody comprising a V_{H} comprising a sequence set forth in SEQ ID NO: 13 and a V_{L} comprising a sequence set forth in SEQ ID NO: 11; and
(ii) a second binding region comprising:
   (a) Annexin A5 comprising a sequence set forth in SEQ ID NO: 14;
   (b) truncated Annexin A1 comprising a sequence set forth in SEQ ID NO: 30;
   (c) a phosphatidylserine binding fragment or variant of Annexin A5; or
   (d) a phosphatidylserine binding fragment or variant of Annexin A1.

In one example, the present disclosure provides a membrane targeted binding protein that binds to a coagulation factor, wherein the protein comprises:
(i) a first binding region that is an anti-Factor IX half antibody comprising a V_{H} comprising a sequence set forth in SEQ ID NO: 13 and a V_{L} comprising a sequence set forth in SEQ ID NO: 11; and
(ii) a second binding region comprising Annexin A5 comprising a sequence set forth in SEQ ID NO: 14.

In one example, the present disclosure provides a membrane targeted binding protein that binds to a coagulation factor, wherein the protein comprises:
(i) a first binding region that is an anti-Factor IX half antibody comprising a V_{H} comprising a sequence set forth in SEQ ID NO: 13 and a V_{L} comprising a sequence set forth in SEQ ID NO: 11; and
(ii) a second binding region comprising truncated Annexin A1 comprising a sequence set forth in SEQ ID NO: 30.

In one example, the present disclosure provides a membrane targeted binding protein that binds to a coagulation factor, wherein the protein comprises:
(i) a first binding region that is an anti-Factor IX half antibody comprising a V_{H} comprising a sequence set forth in SEQ ID NO: 13 and a V_{L} comprising a sequence set forth in SEQ ID NO: 11; and
(ii) a second binding region comprising a phosphatidylserine binding fragment or variant of Annexin A5.

In one example, the present disclosure provides a membrane targeted binding protein that binds to a coagulation factor, wherein the protein comprises:
(i) a first binding region that is an anti-Factor IX half antibody comprising a V_{H} comprising a sequence set forth in SEQ ID NO: 13 and a V_{L} comprising a sequence set forth in SEQ ID NO: 11; and
(ii) a second binding region comprising a phosphatidylserine binding fragment or variant of Annexin A1.

In one example, the present disclosure provides a membrane targeted binding protein that binds to a coagulation factor, wherein the protein comprises:
(i) a first binding region that is an anti-Factor IX half antibody comprising a V_{H} comprising a sequence set forth in SEQ ID NO: 35 and a V_{L} comprising a sequence set forth in SEQ ID NO: 11; and
(ii) a second binding region comprising Annexin A5 comprising a sequence set forth in SEQ ID NO: 14.

In one example, the present disclosure provides a membrane targeted binding protein that binds to a coagulation factor, wherein the protein comprises:
(i) a first binding region that is an anti-Factor IX half antibody comprising a V_{H} comprising a sequence set forth in SEQ ID NO: 38 and a V_{L} comprising a sequence set forth in SEQ ID NO: 11; and
(ii) a second binding region comprising Annexin A5 comprising a sequence set forth in SEQ ID NO: 14.

In one example, the present disclosure provides a membrane targeted binding protein that binds to a coagulation factor, wherein the protein comprises:
(i) a first binding region that is an anti-Factor IX half antibody comprising a V_{H} comprising a sequence set forth in SEQ ID NO: 41 and a V_{L} comprising a sequence set forth in SEQ ID NO: 11; and
(ii) a second binding region comprising Annexin A5 comprising a sequence set forth in SEQ ID NO: 14.

In one example, the present disclosure provides a membrane targeted binding protein that binds to a coagulation factor, wherein the protein comprises:
(i) a first binding region that is an anti-Factor IX half antibody comprising a V_{H} comprising a sequence set forth in SEQ ID NO: 50 and a V_{L} comprising a sequence set forth in SEQ ID NO: 11; and
(ii) a second binding region comprising Annexin A5 comprising a sequence set forth in SEQ ID NO: 14.

In one example, the amino acid sequence of V_{H} comprises a Tyrosine (T), Isoleucine (I) or Lysine (K) or Glutamic Acid (E) at position 103 and/or Lysine (K) or Tyrosine (Y) at position 104 and/or Proline (P), Threonine (T) or Glycine (G) at position 105 and/or Tryptophan (W) or Glycine (G) at position 106 and/or Glycine (G) or Histidine (H) at position 107 and/or Tyrosine (Y) or Phenylalanine (F) or Tryptophan (W) at position 108.

In one example, the amino acid sequence of V_{H} comprises a Glutamic Acid (E) at position 103, Tyrosine (Y) at position 104, Glycine (G) at position 105, Glycine (G) at position 106, Glycine (G) at position 107 and Tryptophan (W) at position 108.

In one example, the amino acid sequence of V_{H} comprises Tyrosine (T) at position 103, Lysine (K) at position 104, Proline (P) at position 105, Tryptophan (W) at position 106, Glycine (G) at position 107 and Tyrosine (Y) at position 108.

In one example, the amino acid sequence of V_{H} comprises Isoleucine (I) at position 103, Lysine (K) at position 104, Threonine (T) at position 105, Tryptophan (W) at position 106, Glycine (G) at position 107 and Tyrosine (Y) at position 108.

In one example, the amino acid sequence of V_{H} comprises Lysine (K) at position 103, Lysine (K) at position 104, Glycine (G) at position 105, Tryptophan (W) at position 106, Histidine (H) at position 107 and Phenylalanine (F) at position 108.

In one example, the first binding region of the membrane targeted binding protein of the present disclosure is linked to the second binding region directly (i.e., without a linking region). In another example, the first binding region is linked to the second binding region via a linker.

In one example, the first binding region and second binding region (and linker, if present) are a fusion protein. Thus, the first binding region and second binding region are covalently linked by an amide bond. The present disclosure encompasses other forms of covalent and non-covalent linkages. For example, the regions can be linked by a chemical linker.

In one example, the linker is a flexible linker, e.g., a flexible peptide linker. For example, the first binding region is linked to the second binding region via a flexible linker.

In one example, the linker is a peptide linker. For example, the first binding region is linked to the second binding region via a linker wherein the linker is a peptide linker comprising between 2 and 31 amino acids in length. For example, the linker sequence is about 16 amino acids in length. In one example, the linker comprises the sequence (Gly₄Ser)₃ or SGGGGSGGGGSGGGGS (GS16) or a sequence set forth in SEQ ID NO: 20. In another example, the linker comprises the sequence SG (GS2) or SGGGGS (GS6) or a sequence set forth in SEQ ID NO: 24. In a further example, the linker comprises the sequence SGGGGSGGGGSGGGGSGGGGSGGGGSGGGGS (GS31) or a sequence set forth in SEQ ID NO: 25.

In one example, the linker is a rigid linker. For example, the rigid linker comprises the sequence (EAAAK)ₙ, where n is between 1 and 3. In one example, the rigid linker comprises the (EAAAK)ₙ, where n is between 1 and 10 or between about 1 and 100. For example, n is at least 1, or at least 2, or at least 3, or at least 4, or at least 5, or at least 6, or at least 7, or at least 8, or at least 9, or at least 10. In one example, n is less than 100. For example, n is less than 90, or less than about 80, or less than about 60, or less than about 50, or less than about 40, or less than about 30, or less than about 20, or less than about 10.

In one example, the linker is a cleavable linker. For example, the linker can be cleaved by a protease or peptidase.

In one example, the linker joins the N-terminus of the second binding region to the N- or C-terminus of a heavy chain or domain thereof (e.g., V_{H}) or a light chain or domain thereof (e.g., V_{L}, CH1) of a first binding region which is an antibody (e.g., an anti-Factor IX antibody or antigen binding fragment thereof). For example, the linker extends between the N-terminus of the second binding region and the C-terminus of a heavy chain or domain thereof of the first binding region (e.g., the anti-Factor IX antibody or antigen binding fragment thereof).

In one example, the flexible linker joins the N-terminus of the second binding region to the C-terminus of a heavy chain or domain thereof (e.g., V_{H}) or a light chain or domain thereof (e.g., V_{L}) of a first binding region which is an antibody (e.g., an anti-Factor IX antibody or antigen binding fragment thereof). In one example, the flexible linker joins the C-terminus of the second binding region to the N-terminus of a heavy chain or domain thereof (e.g., V_{H}) or a light chain or domain thereof (e.g., V_{L}) of a first binding region which is an antibody (e.g., an anti-Factor IX antibody or antigen binding fragment thereof).

In one example, the membrane targeted binding protein of the present disclosure is conjugated to a compound, which is directly or indirectly bound to the membrane targeted binding protein.

In one example, the compound is a therapeutic agent or a detectable agent. Suitable therapeutic agents or detectable agents are known in the art and include, but are not limited to, the group consisting of a cytotoxin, a radioisotope, an immunomodulatory agent, and anti-angiogenic agent, an anti-neovascularisation agent, a toxin, an anti-proliferative agent, a pro-apoptotic agent, a chemotherapeutic agent, a therapeutic nucleic acid and a fluorescent label.

The present disclosure also provides a composition comprising a membrane targeted binding protein of the disclosure and a pharmaceutically acceptable carrier. In one example, the binding protein has pro-coagulant activity. In one example, the binding protein has anti-coagulant activity.

The present disclosure also provides a method of treating or preventing a disease or condition in a subject, the method comprising administering a membrane targeted binding protein of the present disclosure or the composition comprising a membrane targeted binding protein of the present disclosure to a subject in need thereof.

In one example, the present disclosure provides use of a membrane targeted binding protein of the present disclosure in the manufacture of a medicament for the treatment or prevention of a disease or condition in a subject.

In one example, the disease or condition is a bleeding disorder.

In one example, the subject suffers from a bleeding disorder. In one example, the subject has been diagnosed as suffering from a bleeding disorder. In one example, the subject is receiving treatment for a bleeding disorder.

In one example, the subject suffers from a bleeding disorder and has developed inhibitors to a treatment for the bleeding disorder (e.g., has developed inhibitory auto-antibodies against a coagulation factor or a recombinant or modified form thereof).

In one example of any method described herein, the membrane targeted binding protein of the present disclosure is administered before or after the development of a bleeding disorder. In one example of any method described herein, the membrane targeted binding protein of the present disclosure is administered before the development of the bleeding disorder. In one example of any method described herein, the membrane targeted binding protein of the present disclosure is administered after the development of the bleeding disorder.

In one example of any method described herein, the membrane targeted binding protein of the present disclosure is administered before or after the onset of a bleeding event. In one example, the membrane targeted binding protein of the present disclosure is administered before the onset of a bleeding event. In another example, the membrane targeted binding protein of the present disclosure is administered after the onset of a bleeding event.

A bleeding event will be apparent to the skilled person and include, for example a minor and/or major bleeding event. In one example, the bleeding event is a major bleeding event. For example, a major bleeding event is any episode of bleeding that leads to ≥5 g/dL reduced haemoglobin or a ≥15% absolute decrease in haematocrit. In one example, the bleeding event is a minor bleeding event. For example, a minor bleeding event is any episode of bleeding that leads to ≤4 g/dL reduced haemoglobin or a ≥10% absolute decrease in haematocrit.

In one example of any method described herein, the membrane targeted binding protein of the present disclosure is administered after development of inhibitors of a treatment for a bleeding disorder.

In one example, the subject is at risk of developing a bleeding disorder. For example, a subject at risk of developing a bleeding disorder includes, but is not limited, to those with a mutation, deletion or rearrangement in a blood coagulation factor, e.g., factor VIII, or those with a platelet disorder. In one example, the subject has a relative that has developed a bleeding disorder. For example, the bleeding disorder is inherited. In one example, the bleeding disorder is acquired. In one example, a subject at risk of developing a bleeding disorder has developed an inhibitor of a coagulation factor.

In one example, the membrane targeted binding protein is administered before or after the onset of symptoms of a bleeding disorder. In one example, the membrane targeted binding protein is administered before the onset of symptoms of a bleeding disorder. In one example, the membrane targeted binding protein is administered after the onset of symptoms of a bleeding disorder. In one example, the membrane targeted binding protein of the present disclosure is administered at a dose that alleviates or reduces one or more of the symptoms of a bleeding disorder.

Symptoms of a bleeding disorder will be apparent to the skilled person and include, for example:
Easy bruising;
Bleeding gums;
Heavy bleeding from small cuts or dental work;
Unexplained nosebleeds;
Heavy menstrual bleeding;
Bleeding into joints; and/or
Excessive bleeding following surgery.

In one example, the bleeding disorder is caused by a blood coagulation disorder. For example, the blood coagulation disorder is haemophilia, von Willebrand disease, factor I deficiency, factor II deficiency, factor V deficiency, combined factor V/factor VIII deficiency, factor VII deficiency, factor X deficiency, factor XI deficiency or factor XIII deficiency. In one example, the haemophilia is haemophilia A or haemophilia B. In one example, the subject has a condition requiring prophylactic treatment.

In one example, the subject has developed inhibitors (e.g., inhibitory antibodies) of factor VIII.

In one example, the subject suffers from haemophilia A. In one example, the subject suffers from haemophilia A and has developed inhibitor (e.g., inhibitory antibodies) to factor VIII. For example, the protein of the disclosure has factor VIII bypassing activity.

In one example, the membrane targeted binding protein of the present disclosure is administered to the subject in an amount to reduce the severity of the bleeding in the subject. For example, the activity of a coagulation factor is increased or bypassed. For example, the level of coagulation in the subject is increased relative to before treatment with the protein of the disclosure.

In one example of any method described herein, the subject is a mammal, for example a primate such as a human.

Methods of treatment described herein can additionally comprise administering a further compound to reduce, treat or prevent the effect of the bleeding disorder.

The present disclosure also provides a composition comprising a membrane targeted binding protein that binds to a blood coagulation factor for use in treating or preventing a bleeding disorder.

The present disclosure also provides use of a composition comprising a membrane targeted binding protein that binds to a blood coagulation factor in the manufacture of a medicament for treating or preventing a bleeding disorder.

The present disclosure also provides a kit comprising at least one membrane targeted binding protein that binds to a blood coagulation factor packaged with instructions for use in treating or preventing a bleeding disorder in a subject. Optionally, the kit additionally comprises a therapeutically active compound or drug.

The present disclosure also provides a kit comprising at least one membrane targeted binding protein that binds to a blood coagulation factor packaged with instructions to administer the membrane targeted binding protein to a subject who is suffering from or at risk of suffering from a bleeding disorder, optionally, in combination with a therapeutically active compound or drug.

Exemplary effects of membrane targeted binding proteins that bind to a blood coagulation factor are described herein and are to be taken to apply mutatis mutandis to the examples of the disclosure set out in the previous five paragraphs.

The present disclosure also provides methods for inhibiting coagulation comprising administering to a subject in need thereof a protein of the disclosure comprising a first binding region that inhibits a coagulation factor.

### BRIEF DESCRIPTION OF THE DRAWINGS

FIG. 1 is a graphical representation showing the binding affinity of (A) a membrane targeted anti-Factor IX monospecific antibody comprising two Annexin A5 molecules and (B) a membrane targeted anti-Factor IX/X bispecific antibody comprising one Annexin A5 molecule, to phospholipid vesicles.

FIG. 2 is a graphical representation showing the Factor VIII bypassing activity of (A) membrane targeted and non-membrane targeted anti-Factor IX monospecific antibodies; and (B) membrane targeted and non-membrane targeted anti-Factor IX half antibodies.

FIG. 3 is a graphical representation showing the Factor VIII bypassing activity of membrane targeted and non-membrane targeted anti-Factor IX/X bispecific antibodies.

FIG. 4 is a graphical representation showing the Factor VIII bypassing activity of an Annexin A5 membrane targeted anti Factor IX antibody, an E5 mutant Annexin A5 membrane targeted anti Factor IX antibody and a truncated Annexin A1 membrane targeted anti Factor IX antibody.

FIG. 5 is a graphical representation showing Factor VIII bypassing activity of a membrane targeted anti human Factor IX antibody in FVIII-deficient mouse plasma spiked with human FIX measured ex vivo in (A) an aPTT assay and (B) a one-stage clotting assay.

FIG. 6 is a graphical representation showing the Factor VIII bypassing activity of Annexin A5 membrane targeted anti-Factor IX antibody, non-membrane targeted anti-Factor IX antibody and a non-membrane targeted anti-Factor IX/X bispecific antibody using a chromogenic assay in the absence of phospholipids.

FIG. 7 is a graphical representation showing (A) thrombin generation measured in a single experiment representative of three independent experiments (B) thrombin peak height (mean±SD, n=3) and (C) lagtime (mean±SD, n=3) of membrane targeted anti-Factor IX antibodies, a non-membrane targeted anti-Factor IX/X bispecific antibody and controls in an intrinsic coagulation assay in human FVIII depleted plasma.

### KEY TO SEQUENCE LISTING

SEQ ID NO: 1 amino acid sequence of anti-Factor X/Factor IX antibody light chain
SEQ ID NO: 2 amino acid sequence of anti-Factor X antibody heavy chain
SEQ ID NO: 3 amino acid sequence of anti-Factor IX antibody heavy chain
SEQ ID NO: 4 amino acid sequence of anti-Factor X antibody heavy chain
SEQ ID NO: 5 amino acid sequence of anti-Factor IX antibody heavy chain
SEQ ID NO: 6 amino acid sequence of anti-Factor IX antibody heavy chain
SEQ ID NO: 7 amino acid sequence of anti-Factor X antibody heavy chain
SEQ ID NO: 8 amino acid sequence of anti-Factor IX antibody heavy chain fused to Annexin A5
SEQ ID NO: 9 amino acid sequence of anti-Factor IX antibody heavy chain fused to Annexin A5
SEQ ID NO: 10 amino acid sequence of anti-Factor X heavy chain fused to Annexin A5
SEQ ID NO: 11 light chain V_{L} amino acid sequence of anti-Factor X/Factor IX antibody light chain
SEQ ID NO: 12 heavy chain V_{H} amino acid sequence of anti-Factor X antibody
SEQ ID NO: 13 heavy chain V_{H} amino acid sequence of anti-Factor IX antibody
SEQ ID NO: 14 amino acid sequence of wild-type Annexin A5
SEQ ID NO: 15 amino acid sequence of human IgG4 heavy chain constant region with S228P mutation
SEQ ID NO: 16 amino acid sequence of human IgG4 heavy chain constant region with S228P, T366W mutations
SEQ ID NO: 17 amino acid sequence of human IgG4 heavy chain constant region with S228P, T366S, L368A, Y407V mutations
SEQ ID NO: 18 amino acid sequence of human IgG4 heavy chain constant region with T350V, T366L, K392L, T394W mutations
SEQ ID NO: 19 amino acid sequence of human IgG4 heavy chain constant region with T350V, L351Y, F405A, Y407V mutations
SEQ ID NO: 20 amino acid sequence of a linker GS16
SEQ ID NO: 21 amino acid sequence of human coagulation Factor VIII
SEQ ID NO: 22 amino acid sequence of human coagulation Factor IX
SEQ ID NO: 23 amino acid sequence of human coagulation Factor X
SEQ ID NO: 24 amino acid sequence of a linker GS6
SEQ ID NO: 25 amino acid sequence of a linker GS31
SEQ ID NO: 26 amino acid sequence of E5-mutant of Annexin A5
SEQ ID NO: 27 amino acid sequence of human Lactadherin C1C2 sequence (also known as MFG-E8)
SEQ ID NO: 28 Amino acid sequence of PSP1
SEQ ID NO: 29 amino acid sequence of wild-type Annexin A1
SEQ ID NO: 30 amino acid sequence of truncated Annexin A1
SEQ ID NO: 31 amino acid sequence of anti-Factor IX antibody heavy chain fused to E5-mutant of Annexin A5
SEQ ID NO: 32 amino acid sequence of anti-Factor IX antibody heavy chain fused to truncated Annexin A1
SEQ ID NO: 33 amino acid sequence of anti-Factor IX antibody heavy chain fused to truncated Annexin A1
SEQ ID NO: 34 amino acid sequence of anti-Factor IX antibody heavy chain A10
SEQ ID NO: 35 heavy chain V_{H} amino acid sequence of anti-Factor IX antibody A10
SEQ ID NO: 36 amino acid sequence of anti-Factor IX antibody heavy chain A10 fused to annexin A5
SEQ ID NO: 37 amino acid sequence of anti-Factor IX antibody heavy chain B2
SEQ ID NO: 38 heavy chain V_{H} amino acid sequence of anti-Factor IX antibody B2
SEQ ID NO: 39 amino acid sequence of anti-Factor IX antibody heavy chain B2 fused to Annexin A5
SEQ ID NO: 40 amino acid sequence of anti-Factor IX antibody heavy chain C12
SEQ ID NO: 41 heavy chain V_{H} amino acid sequence of anti-Factor IX antibody C12
SEQ ID NO: 42 amino acid sequence of anti-Factor IX antibody heavy chain C12 fused to Annexin A5
SEQ ID NO: 43 heavy chain V_{H} CDR1 amino acid sequence of anti-Factor IX antibody
SEQ ID NO: 44 heavy chain V_{H} CDR2 amino acid sequence of anti-Factor IX antibody
SEQ ID NO: 45 heavy chain V_{H} CDR3 amino acid sequence of anti-Factor IX antibody
SEQ ID NO: 46 heavy chain V_{H} CDR3 amino acid consensus sequence of anti-Factor IX antibody
SEQ ID NO: 47 light chain V_{L} CDR1 amino acid sequence of anti-Factor IX antibody
SEQ ID NO: 48 light chain V_{L} CDR2 amino acid sequence of anti-Factor IX antibody
SEQ ID NO: 49 light chain V_{L} CDR3 amino acid sequence of anti-Factor IX antibody
SEQ ID NO: 50 heavy chain V_{H} amino acid consensus sequence of anti-Factor IX antibody
SEQ ID NO: 51 heavy chain V_{H} CDR3 amino acid sequence of anti-Factor IX antibody A10
SEQ ID NO: 52 heavy chain V_{H} CDR3 amino acid sequence of anti-Factor IX antibody B2
SEQ ID NO: 53 heavy chain V_{H} CDR3 amino acid sequence of anti-Factor IX antibody C12
SEQ ID NO: 54 amino acid sequence of anti-Factor IX antibody heavy chain ATG16028
SEQ ID NO: 55 amino acid sequence of anti-Factor IX antibody heavy chain ATG16028 fused to Annexin A5

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, antibodies and fragments thereof herein may be further clarified by the discussion in Kabat Sequences of Proteins of Immunological Interest, National Institutes of Health, Bethesda, Md., 1987 and 1991.

The term “EU numbering system of Kabat” will be understood to mean the numbering of an antibody heavy chain is according to the EU index as taught in Kabat et al., 1991, Sequences of Proteins of Immunological Interest, 5th Ed., United States Public Health Service, National Institutes of Health, Bethesda. The EU index is based on the residue numbering of the human IgG1 EU antibody.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

Reference herein to a range of, e.g., residues, will be understood to be inclusive. For example, reference to “a region comprising amino acids 56 to 65 of SEQ ID NO: 1” will be understood to mean that the region comprises a sequence of amino acids as numbered 56, 57, 58, 59, 60, 61, 62, 63, 64 and 65 in SEQ ID NO: 1.

### Selected Definitions

As used herein, the term “membrane targeted” refers to a protein that binds to a component of a plasma membrane of a mammalian cell. For example, the mammalian cell comprises structurally defined domains on the plasma membrane that associate with the protein.

As used herein, “coagulation factor” refers to a factor that is associated with the formation of a blot clot, i.e., blood coagulation. In one example, the coagulation factor has pro-coagulant activity. Coagulation factors are known in the art and include without limitation factor I, factor II, factor III, factor V, factor VII, factor VIII, factor IX, factor X, factor XI, factor XII and factor XIII or an activated form of any of the foregoing. This term also includes recombinant forms of coagulation factors and/or modified forms thereof, e.g., as is known in the art and/or described herein.

The term “distinct” in the context of coagulation factors refers to two or more coagulation factors that are distinguishable or different from each other. For example, the two or more coagulation factors are not identical to each other e.g., factor IX and factor X.

“Pro-coagulant activity” refers to an effect of enhancing or promoting the coagulation of the blood. In some examples, binding of a membrane targeted binding protein to a coagulation factor may not directly cause coagulation, but may play a role in the coagulation cascade by facilitating/enhancing a coagulation reaction. For example, the level of the reaction (which could be activation of a coagulation factor or level of coagulation) is enhanced in the presence of the membrane targeted binding protein compared to in the absence of the protein. Thus, in some examples, a membrane targeted binding protein does not have any coagulation activity in its own right, e.g., the membrane targeted binding protein facilitates a reaction in the coagulation cascade or facilitates coagulation. In some examples, binding of a membrane targeted binding protein to an activated coagulation factor (e.g. activated FIX) may stabilize this factor in its active conformation. Without being bound by theory or mode of action, in the case of Factor IX, such stabilization may enhance its catalytic cofactor activity for intrinsic activation of the coagulation pathway. “Pro-coagulant activity” may be “bypassing activity”.

“Anti-coagulant activity” refers to an effect of retarding or inhibiting the coagulation of the blood. Binding of a membrane targeted binding protein to a coagulation factor may not directly inhibit coagulation but may play an essential role in slowing or inhibiting the coagulation cascade.

As used herein, the term “bypassing activity” refers to the ability of a membrane targeted binding protein to bypass or substitute for an endogenous coagulation factor in the coagulation cascade. For example, the binding region of the membrane targeted binding protein substitutes for an endogenous coagulation factor in the coagulation cascade, e.g., the intrinsic coagulation cascade. For example, the membrane targeted binding protein has the ability to mimic or substitute for coagulation-enhancing properties of a missing (e.g., non-expressed), non-functional (e.g., mutant) or blocked (e.g., by inhibitors) coagulation factor, for example by increasing the pro-coagulant activity of an upstream coagulation factor or by replacing a missing or non-functional coagulation factor such that the missing, non-functional or blocked endogenous coagulation factor is no longer required for effective thrombin generation or coagulation activity.

The term “binding region” shall be understood to refer to a membrane targeted binding protein or part thereof or other region of the membrane targeted binding protein that is capable of interacting with or specifically binding to an antigen (e.g., a cell component or molecule, such as a protein, e.g., a coagulation factor). For example, the binding region can be an antibody or a half-antibody or an antigen binding fragment of an antibody (e.g., a Fv or a scFv or a diabody, etc.)

As used herein, the term “binds” in reference to the interaction of a binding region of a membrane targeted binding protein with a component (i.e., blood coagulation factor or a component of a plasma membrane) means that the interaction is dependent upon the presence of a particular structure (e.g., epitope) on the component. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the protein, will reduce the amount of labeled “A” bound to the antibody.

As used herein, the term “specifically binds” shall be taken to mean that the binding interaction between the binding region on the membrane targeted binding protein and component (i.e., blood coagulation factor or component of a plasma membrane) is dependent on the presence of the antigenic determinant or epitope. The binding region preferentially binds or recognizes a specific antigenic determinant or epitope even when present in a mixture of other molecules or organisms. In one example, the binding region reacts or associates more frequently, more rapidly, with greater duration and/or with greater affinity with the specific component or cell expressing same than it does with alternative antigens or cells. It is also understood by reading this definition that, for example, a binding region the specifically binds to a particular component may or may not specifically bind to a second antigen. As such, “specific binding” does not necessarily require exclusive binding or non-detectable binding of another antigen. The term “specifically binds” can be used interchangeably with “selectively binds” herein. Generally, reference herein to binding means specific binding, and each term shall be understood to provide explicit support for the other term. Methods for determining specific binding will be apparent to the skilled person. For example, a binding protein comprising the binding region of the disclosure is contacted with the component or a cell expressing same or a mutant form thereof or an alternative antigen. The binding to the component or mutant form or alternative antigen is then determined and a binding region that binds as set out above is considered to specifically bind to the component. In one example, “specific binding” to the component or cell expressing same, means that the binding region binds with an equilibrium constant (K_{D}) of 1 μM or less, such as 100 nM or less, such as 50 nM or less, for example 20 nM or less, such as, 1 nM or less, e.g., 0.8 nM or less, 1×10⁻⁸M or less, such as 5×10⁻⁹M or less, for example, 3×10⁻⁹M or less, such as 2.5×10⁻⁹M or less.

The term “preferentially binds” shall be taken to mean that a binding region on the membrane targeted binding protein binds to one component (i.e., blood coagulation factor or component of a plasma membrane) in preference to, or in favour of, another component. As such, “preferential binding” does not necessarily require exclusive binding or non-detectable binding of another component. For example, the membrane targeted binding protein of the present disclosure preferentially binds to activated factor IXa compared to the non-activated factor FIX.

The term “component of a plasma membrane” shall be understood to mean any component that is present on the surface of a mammalian cell to which a binding region of a membrane targeted binding protein may bind. In one example, the component is exposed on the extracellular surface of the plasma membrane of the cell. In one example, the component may be present abundantly on the surface of the mammalian cell to enable specific and efficient targeting of the binding protein. For example, the component may be present in an amount sufficient for binding in vivo to initiate an effect following binding of the binding region. Examples of components that are present on the surface of mammalian cells are known in the art and include, but are not limited to, aminophospholipids (e.g., phosphatidylserines or phosphatidylethanolamine); membrane-associated polypeptides (e.g., glycoproteins GPIIb/IIIa, β2GP1, TLT-1, coagulation factors and selectins), and membrane-associated complexes comprising two or more distinct coagulation factors.

The term “recombinant” shall be understood to mean the product of artificial genetic recombination. Accordingly, in the context of an antibody or antigen binding fragment thereof, this term does not encompass an antibody naturally occurring within a subject's body that is the product of natural recombination that occurs during B cell maturation. However, if such an antibody is isolated, it is to be considered an isolated protein comprising an antibody variable region. Similarly, if nucleic acid encoding the protein is isolated and expressed using recombinant means, the resulting protein is a recombinant protein. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

The skilled artisan will be aware that an “antibody” is generally considered to be a protein that comprises a variable region made up of a plurality of polypeptide chains, e.g., a polypeptide comprising a light chain variable region (V_{L}) and a polypeptide comprising a heavy chain variable region (V_{H}). An antibody also generally comprises constant domains, some of which can be arranged into a constant region, which includes a constant fragment or fragment crystallizable (Fc), in the case of a heavy chain. A V_{H} and a V_{L} interact to form an Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies, synhumanized antibodies and chimeric antibodies. The term “antibody” also includes variants missing an encoded C-terminal lysine residue, a deamidated variant and/or a glycosylated variant and/or a variant comprising a pyroglutamate, e.g., at the N-terminus of a protein (e.g., antibody) and/or a variant lacking a N-terminal residue, e.g., a N-terminal glutamine in an antibody or V region and/or a variant comprising all or part of a secretion signal. Deamidated variants of encoded asparagine residues may result in isoaspartic, and aspartic acid isoforms being generated or even a succinamide involving an adjacent amino acid residue. Deamidated variants of encoded glutamine residues may result in glutamic acid. Compositions comprising a heterogeneous mixture of such sequences and variants are intended to be included when reference is made to a particular amino acid sequence.

In the context of the present disclosure, the term “half antibody” refers to a protein comprising a single antibody heavy chain and a single antibody light chain. The term “half antibody” also encompasses a protein comprising an antibody light chain and an antibody heavy chain, wherein the antibody heavy chain has been mutated to prevent association with another antibody heavy chain.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that specifically binds to an antigen and, for example, includes amino acid sequences of CDRs; i.e., CDR1, CDR2, and CDR3, and framework regions (FRs). For example, the variable region comprises three or four FRs (e.g., FR1, FR2, FR3 and optionally FR4) together with three CDRs. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region typically has three CDR regions identified as CDR1, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”. According to the numbering system of Kabat, V_{H} FRs and CDRs are positioned as follows: residues 1-30 (FR1), 31-35 (CDR1), 36-49 (FR2), 50-65 (CDR2), 66-94 (FR3), 95-102 (CDR3) and 103-113 (FR4). According to the numbering system of Kabat, V_{L} FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4).

“Framework regions” (hereinafter FR) are those variable domain residues other than the CDR residues.

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an antibody, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

The term “constant region” as used herein, refers to a portion of heavy chain or light chain of an antibody other than the variable region. In a heavy chain, the constant region generally comprises a plurality of constant domains and a hinge region, e.g., a IgG constant region comprises the following linked components, a constant heavy (C_{H})1, a linker, a C_{H}2 and a C_{H}3. In a heavy chain, a constant region comprises a Fc. In a light chain, a constant region generally comprises one constant domain (a C_{L}1).

The term “fragment crystalizable” or “Fc” or “Fc region” or “Fc portion” (which can be used interchangeably herein) refers to a region of an antibody comprising at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Furthermore, heavy chains of various subclasses (such as the IgG subclasses of heavy chains) are responsible for different effector functions and thus, by choosing the desired heavy chain constant region, proteins with desired effector function can be produced. Exemplary heavy chain constant regions are gamma 1 (IgG₁), gamma 2 (IgG₂) and gamma 3 (IgG₃), or hybrids thereof.

An “antigen binding fragment” of an antibody comprises one or more variable regions of an intact antibody. Examples of antibody fragments include Fab, Fab′, F(ab′)₂ and Fv fragments; diabodies; linear antibodies; single-chain antibody molecules, half antibodies and multispecific antibodies formed from antibody fragments.

The term “stabilized IgG₄ constant region” will be understood to mean an IgG₄ constant region that has been modified to reduce Fab arm exchange or the propensity to undergo Fab arm exchange or formation of a half-antibody or a propensity to form a half antibody. “Fab arm exchange” refers to a type of protein modification for human IgG₄, in which an IgG₄ heavy chain and attached light chain (half-molecule) is swapped for a heavy-light chain pair from another IgG₄ molecule. Thus, IgG₄ molecules may acquire two distinct Fab arms recognizing two distinct antigens (resulting in bispecific molecules). Fab arm exchange occurs naturally in vivo and can be induced in vitro by purified blood cells or reducing agents such as reduced glutathione.

As used herein, the term “monospecific” refers to a binding region comprising one or more antigen binding sites each with the same epitope specificity. Thus, a monospecific binding region can comprise a single antigen binding site (e.g., a Fv, scFv, Fab, etc) or can comprise several antigen binding sites that recognize the same epitope (e.g., are identical to one another), e.g., a diabody or an antibody. The requirement that the binding region is “monospecific” does not mean that it binds to only one antigen, since multiple antigens can have shared or highly similar epitopes that can be bound by a single antigen binding site. A monospecific binding region that binds to only one antigen is said to “exclusively bind” to that antigen.

The term “multispecific” refers to a binding region comprising two or more antigen binding sites, each of which binds to a distinct epitope, for example each of which binds to a distinct antigen. For example, the multispecific binding region may include antigen binding sites that recognise two or more different epitopes of the same protein (e.g., coagulation factor) or that may recognise two or more different epitopes of different proteins (i.e., distinct coagulation factors). In one example, the binding region may be “bispecific”, that is, it includes two antigen binding sites that specifically bind two distinct epitopes. For example, a bispecific binding region specifically binds or has specificities for two different epitopes on the same protein. In another example, a bispecific binding region specifically binds two distinct epitopes on two different proteins (e.g., factor IX and factor X).

As used herein, the terms “disease”, “disorder” or “condition” refers to a disruption of or interference with normal function, and is not to be limited to any specific condition, and will include diseases or disorders.

As used herein, the term “bleeding condition” or “bleeding disorder” refers to a condition in which there is abnormal blood coagulation, e.g., reduced or insufficient blood coagulation capability and/or abnormal bleeding (internal and/or external), e.g., excessive bleeding.

As used herein, a subject “at risk” of developing a disease or condition or relapse thereof or relapsing may or may not have detectable disease or symptoms of disease, and may or may not have displayed detectable disease or symptoms of disease prior to the treatment according to the present disclosure. “At risk” denotes that a subject has one or more risk factors, which are measurable parameters that correlate with development of the disease or condition, as known in the art and/or described herein.

As used herein, the terms “treating”, “treat” or “treatment” include administering a protein described herein to thereby reduce or eliminate at least one symptom of a specified disease or condition or to slow progression of the disease or condition.

As used herein, the term “preventing”, “prevent” or “prevention” includes providing prophylaxis with respect to occurrence or recurrence of a bleeding disease or a symptom of a bleeding disease in an individual. An individual may be predisposed to or at risk of developing the disease or disease relapse but has not yet been diagnosed with the disease or the relapse.

An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment of a disease or condition as hereinbefore described. In some examples of the present disclosure, the term “effective amount” is meant an amount necessary to effect a change in a factor associated with a disease or condition as hereinbefore described. For example, the effective amount may be sufficient to effect a change in the level of coagulation. The effective amount may vary according to the disease or condition to be treated or factor to be altered and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. Accordingly, this term is not to be construed to limit the disclosure to a specific quantity, e.g., weight or number of binding proteins. The effective amount can be administered in a single dose or in a dose repeated once or several times over a treatment period.

A “therapeutically effective amount” is at least the minimum concentration required to effect a measurable improvement of a particular disease or condition. A therapeutically effective amount herein may vary according to factors such as the disease state, age, sex, and weight of the patient, and the ability of the antibody or antigen binding fragment thereof to elicit a desired response in the individual. A therapeutically effective amount is also one in which any toxic or detrimental effects of the antibody or antigen binding fragment thereof are outweighed by the therapeutically beneficial effects. In one example, a therapeutically effective amount shall be taken to mean a sufficient quantity of membrane targeted binding protein to reduce or inhibit one or more symptoms of a bleeding disorder or a complication thereof.

As used herein, the term “prophylactically effective amount” shall be taken to mean a sufficient quantity of membrane targeted binding protein to prevent or inhibit or delay the onset of one or more detectable symptoms of a bleeding disorder or a complication thereof.

As used herein, the term “subject” shall be taken to mean any animal including humans, for example a mammal. Exemplary subjects include but are not limited to humans and non-human primates. For example, the subject is a human.

### Coagulation Factors

The present disclosure provides a membrane targeted binding protein that binds at least one coagulation factor.

Blood coagulation occurs through a cascade of stages involving release of several coagulation factors, ultimately resulting in the formation of a blood clot containing insoluble fibrin. Exemplary coagulation factors include, but are not limited to, factor I (Fibrinogen), factor II (Prothrombin/thrombin), factor III (Tissue factor), factor V (Labile factor), factor VII (Proconvertin), factor VIII (Antihaemophilic factor), factor IX (Christmas factor), factor X (Stuart-Prower factor), factor XI (Plasma thromboplastin antecedent), factor XII (Hageman (contact) factor) and factor XIII (Fibrin-stabilizing factor/Prekallikrein (Fletcher) factor/HMWK (Fitzgerald) factor).

In one example, the present disclosure provides a membrane targeted binding protein comprising a first binding region that specifically binds to a coagulation factor. In one example, the coagulation factor is factor VIII. For the purposes of nomenclature only and not limitation, exemplary sequences of human factor VIII are set out in NCBI Ref Seq ID NP_000123, protein accession number NM_000132.3 and in SEQ ID NO: 21.

In one example, the coagulation factor is factor IX. For the purposes of nomenclature only and not limitation, exemplary sequences of human factor IX are set out in GenBank ID AAA98726.1 and in SEQ ID NO: 22.

In one example, the coagulation factor is factor X. For the purposes of nomenclature only and not limitation, exemplary sequences of human factor X are set out in Gene ID: 2159 and in SEQ ID NO: 23.

For the purposes of nomenclature only and not limitation, exemplary sequences of human factor I are set out in NCBI Ref Seq ID NM_000508 (alpha chain) and NM_005141 (beta chain), exemplary sequences of human factor II are set out in Ref Seq ID NM_000506, exemplary sequences of human factor III are set out in Ref Seq ID NM_001993, exemplary sequences of human factor V are set out in Ref Seq ID NM_000130, exemplary sequences of human factor VII are set out in Ref Seq ID NM_00131, exemplary sequences of human factor XI are set out in Ref Seq ID NM_000128, exemplary sequences of human factor XII are set out in Ref Seq ID NM_000505, exemplary sequences of human factor XIII are set out in Ref Seq ID NM_000129 (A chain) and NM_001994 (B chain).

Additional sequence of coagulation factors can be determined using sequences provided herein and/or in publically available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)).

A membrane targeted binding protein of the disclosure can also bind to a recombinant form of a coagulation factor.

A membrane targeted binding protein of the disclosure can also bind to a modified form of a coagulation factor. Modified forms of coagulation factors are known in the art and described for example, in Morfini and Zanon Expert Opinion on Emerging Drugs, 21: 301-313, 2016 or Peyvandi et al., Journal of Thrombosis and Haemostasis, 11: 84-98, 2013. Exemplary modified forms of coagulation factors include, truncated proteins, PEGylated proteins, glycopegylated proteins, Fc fusion proteins, albumin fusion proteins, albumin conjugates, single chain proteins, and mixtures of such modifications. Modified forms of factor VIII include, B domain deleted forms, PEGylated forms, Fc fusion forms, single chains forms and mixtures thereof, such as, Turoctocog alfa, Turoctocog alfa Pegol, Simoctocog alfa, Damoctocog alfa pegol, Octocog alfa pegol, lonoctocog alfa or Efraloctocog alfa. Modified forms of factor IX include, PEGylated forms, Fc fusion forms and albumin fusions, such as, Albutrepenonacog alfa, Eftrenonacog alfa or Nonacog beta pegol.

A membrane targeted binding protein of the disclosure that binds a modified form of a coagulation factor can also bind to the endogenous form thereof and/or an unmodified recombinant form thereof.

### Binding Proteins

As discussed herein, binding proteins of the present disclosure can take various forms and comprise one or more binding regions. An exemplary binding protein of the present disclosure comprises a first binding region that specifically binds to a blood coagulation factor and a second binding region that specifically binds to a component of a plasma membrane of a mammalian cell. Typically, the first binding region of the present disclosure comprises an antibody or antigen-binding fragment thereof. Exemplary binding proteins and binding regions are discussed herein.

### Antibodies

In one example, the membrane targeted binding protein of the present disclosure comprises an antibody or antigen binding fragment thereof.

### Immunization-Based Methods

Methods for generating antibodies are known in the art and/or described in Harlow and Lane (editors) Antibodies: A Laboratory Manual, Cold Spring Harbor Laboratory, (1988). Generally, in such methods a protein or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, sub-cutaneously, intravenously, intradermally, intraperitoneally, or by other known route.

The production of polyclonal antibodies may be monitored by sampling blood of the immunized animal at various points following immunization. One or more further immunizations may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (Mabs).

Monoclonal antibodies are one exemplary form of antibody contemplated by the present disclosure. The term “monoclonal antibody” or “mAb” refers to a homogeneous antibody population capable of binding to the same antigen(s), for example, to the same epitope within the antigen. This term is not intended to be limited as regards to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

For example, a suitable animal is immunized with an immunogen under conditions sufficient to stimulate antibody producing cells. Rodents such as rabbits, mice and rats are exemplary animals. Mice genetically-engineered to express human immunoglobulin proteins and, for example, do not express murine immunoglobulin proteins, can also be used to generate an antibody of the present disclosure (e.g., as described in WO2002066630).

Following immunization, somatic cells with the potential for producing antibodies, e.g., B lymphocytes (B cells), are selected for use in the MAb generating protocol. These cells may be obtained from biopsies of spleens, tonsils or lymph nodes, or from a peripheral blood sample. The B cells from the immunized animal are then fused with cells of an immortal myeloma cell, generally derived from the same species as the animal that was immunized with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by flow cytometry and/or immunohistochemstry and/or immunoassay (e.g. radioimmunoassay, enzyme immunoassay, cytotoxicity assay, plaque assay, dot immunoassay, and the like).

Alternatively, ABL-MYC technology (NeoClone, Madison Wis. 53713, USA) is used to produce cell lines secreting MAbs (e.g., as described in Largaespada et al, J. Immunol. Methods. 197: 85-95, 1996).

### Library-Based Methods

The present disclosure also encompasses screening of libraries of antibodies or antigen binding fragments thereof (e.g., comprising variable regions thereof).

Examples of libraries contemplated by this disclosure include naïve libraries (from unchallenged subjects), immunized libraries (from subjects immunized with an antigen) or synthetic libraries. Nucleic acid encoding antibodies or regions thereof (e.g., variable regions) are cloned by conventional techniques (e.g., as disclosed in Sambrook and Russell, eds, Molecular Cloning: A Laboratory Manual, 3rd Ed, vols. 1-3, Cold Spring Harbor Laboratory Press, 2001) and used to encode and display proteins using a method known in the art. Other techniques for producing libraries of proteins are described in, for example in U.S. Pat. No. 6,300,064 (e.g., a HuCAL library of Morphosys AG); U.S. Pat. Nos. 5,885,793; 6,204,023; 6,291,158; or 6,248,516.

The antigen binding fragments according to the disclosure may be soluble secreted proteins or may be presented as a fusion protein on the surface of a cell, or particle (e.g., a phage or other virus, a ribosome or a spore). Various display library formats are known in the art. For example, the library is an in vitro display library (e.g., a ribosome display library, a covalent display library or a mRNA display library, e.g., as described in U.S. Pat. No. 7,270,969). In yet another example, the display library is a phage display library wherein proteins comprising antigen binding fragments of antibodies are expressed on phage, e.g., as described in U.S. Pat. Nos. 6,300,064; 5,885,793; 6,204,023; 6,291,158; or 6,248,516. Other phage display methods are known in the art and are contemplated by the present disclosure. Similarly, methods of cell display are contemplated by the disclosure, e.g., bacterial display libraries, e.g., as described in U.S. Pat. No. 5,516,637; yeast display libraries, e.g., as described in U.S. Pat. No. 6,423,538 or a mammalian display library.

Methods for screening display libraries are known in the art. In one example, a display library of the present disclosure is screened using affinity purification, e.g., as described in Scopes (In: Protein purification: principles and practice, Third Edition, Springer Verlag, 1994). Methods of affinity purification typically involve contacting proteins comprising antigen binding fragments displayed by the library with a target antigen (e.g., IL-3Rα) and, following washing, eluting those domains that remain bound to the antigen.

Any variable regions or scFvs identified by screening are readily modified into a complete antibody, if desired. Exemplary methods for modifying or reformatting variable regions or scFvs into a complete antibody are described, for example, in Jones et al., J Immunol Methods. 354:85-90, 2010; or Jostock et al., J Immunol Methods, 289: 65-80, 2004; or WO2012040793. Alternatively, or additionally, standard cloning methods are used, e.g., as described in Ausubel et al (In: Current Protocols in Molecular Biology. Wiley Interscience, ISBN 047 150338, 1987), and/or (Sambrook et al (In: Molecular Cloning: Molecular Cloning: A Laboratory Manual, Cold Spring Harbor Laboratories, New York, Third Edition 2001).

### Deimmunized, Chimeric, Humanized, Synhumanized, Primatized and Human Antibodies or Antigen Binding Fragments

The antibodies or antigen binding fragments of the present disclosure may be may be humanized.

The term “humanized antibody” shall be understood to refer to a protein comprising a human-like variable region, which includes CDRs from an antibody from a non-human species (e.g., mouse or rat or non-human primate) grafted onto or inserted into FRs from a human antibody (this type of antibody is also referred to a “CDR-grafted antibody”). Humanized antibodies also include antibodies in which one or more residues of the human protein are modified by one or more amino acid substitutions and/or one or more FR residues of the human antibody are replaced by corresponding non-human residues. Humanized antibodies may also comprise residues which are found in neither the human antibody or in the non-human antibody. Any additional regions of the antibody (e.g., Fc region) are generally human. Humanization can be performed using a method known in the art, e.g., U.S. Pat. No. 5,225,539, 6,054,297, 7,566,771 or 5,585,089. The term “humanized antibody” also encompasses a super-humanized antibody, e.g., as described in U.S. Pat. No. 7,732,578. A similar meaning will be taken to apply to the term “humanized antigen binding fragment”.

The antibodies or antigen binding fragments thereof of the present disclosure may be human antibodies or antigen binding fragments thereof. The term “human antibody” as used herein refers to antibodies having variable and, optionally, constant antibody regions found in humans, e.g. in the human germline or somatic cells or from libraries produced using such regions. The “human” antibodies can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4 or 5 of the residues of the protein). These “human antibodies” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies. For the purposes of the present disclosure, a human antibody will also be considered to include a protein comprising FRs from a human antibody or FRs comprising sequences from a consensus sequence of human FRs and in which one or more of the CDRs are random or semi-random, e.g., as described in U.S. Pat. No. 6,300,064 and/or 6,248,516. A similar meaning will be taken to apply to the term “human antigen binding fragment”.

The antibodies or antigen binding fragments thereof of the present disclosure may be synhumanized antibodies or antigen binding fragments thereof. The term “synhumanized antibody” refers to an antibody prepared by a method described in WO2007019620. A synhumanized antibody includes a variable region of an antibody, wherein the variable region comprises FRs from a New World primate antibody variable region and CDRs from a non-New World primate antibody variable region.

The antibody or antigen binding fragment thereof of the present disclosure may be primatized. A “primatized antibody” comprises variable region(s) from an antibody generated following immunization of a non-human primate (e.g., a cynomolgus macaque). Optionally, the variable regions of the non-human primate antibody are linked to human constant regions to produce a primatized antibody. Exemplary methods for producing primatized antibodies are described in U.S. Pat. No. 6,113,898.

In one example an antibody or antigen binding fragment thereof of the disclosure is a chimeric antibody or fragment. The term “chimeric antibody” or “chimeric antigen binding fragment” refers to an antibody or fragment in which one or more of the variable domains is from a particular species (e.g., murine, such as mouse or rat) or belonging to a particular antibody class or subclass, while the remainder of the antibody or fragment is from another species (such as, for example, human or non-human primate) or belonging to another antibody class or subclass. In one example, a chimeric antibody comprising a V_{H} and/or a V_{L} from a non-human antibody (e.g., a murine antibody) and the remaining regions of the antibody are from a human antibody. The production of such chimeric antibodies and antigen binding fragments thereof is known in the art, and may be achieved by standard means (as described, e.g., in U.S. Pat. Nos. 6,331,415; 5,807,715; 4,816,567 and 4,816,397).

The present disclosure also contemplates a deimmunized antibody or antigen binding fragment thereof, e.g., as described in WO2000034317 and WO2004108158. De-immunized antibodies and fragments have one or more epitopes, e.g., B cell epitopes or T cell epitopes removed (i.e., mutated) to thereby reduce the likelihood that a subject will raise an immune response against the antibody or protein. For example, an antibody of the disclosure is analyzed to identify one or more B or T cell epitopes and one or more amino acid residues within the epitope is mutated to thereby reduce the immunogenicity of the antibody.

### Bispecific Antibodies

The antibodies or antigen binding fragments of the present disclosure may be bispecific antibodies or fragments thereof. For example, the antibody or fragment may bind to two or more blood coagulation factors. In another example, the bispecific antibody or fragment can bind to a blood coagulation factor and to a component of a plasma membrane of a mammalian cell. A bispecific antibody is a molecule comprising two types of antibodies or antibody fragments (e.g., two half antibodies) having specificities for different antigens or epitopes. Exemplary bispecific antibodies bind to two different epitopes of the same protein. Alternatively, the bispecific antibody binds to two different epitopes on two different proteins.

Exemplary “key and hole” or “knob and hole” bispecific proteins as described in U.S. Pat. No. 5,731,168. In one example, a constant region (e.g., an IgG₄ constant region) comprises a T366W mutation (or knob) and a constant region (e.g., an IgG₄ constant region) comprises a T366S, L368A and Y407V mutation (or hole). In another example, the first constant region comprises T350V, T366L, K392L and T394W mutations (knob) and the second constant region comprises T350V, L351Y, F405A and Y407V mutations (hole).

Methods for generating bispecific antibodies are known in the art and exemplary methods are described herein.

In one example, an IgG type bispecific antibody is secreted by a hybrid hybridoma (quadroma) formed by fusing two types of hybridomas that produce IgG antibodies (Milstein C et al., Nature 1983, 305: 537-540). In another example, the antibody can be secreted by introducing into cells genes of the L chains and H chains that constitute the two IgGs of interest for co-expression (Ridgway, J B et al. Protein Engineering 1996, 9: 617-621; Merchant, A M et al. Nature Biotechnology 1998, 16: 677-681).

In one example, a bispecific antibody fragment is prepared by chemically cross-linking Fab's derived from different antibodies (Keler T et al. Cancer Research 1997, 57: 4008-4014).

In one example, a leucine zipper derived from Fos and Jun or the like is used to form a bispecific antibody fragment (Kostelny S A et al. J. of Immunology, 1992, 148: 1547-53).

In one example, a bispecific antibody fragment is prepared in a form of diabody comprising two crossover scFv fragments (Holliger P et al. Proc. of the National Academy of Sciences of the USA 1993, 90: 6444-6448).

Multispecific proteins can also be prepared that bind to two or more blood coagulation factors and to a component of a plasma membrane of a mammalian cell, e.g., a trispecific molecule.

### Antibody Fragments

### Single-Domain Antibodies

In some examples, an antigen binding fragment of an antibody of the disclosure is or comprises a single-domain antibody (which is used interchangeably with the term “domain antibody” or “dAb”). A single-domain antibody is a single polypeptide chain comprising all or a portion of the heavy chain variable domain of an antibody.

### Diabodies, Triabodies, Tetrabodies

In some examples, an antigen binding fragment of the disclosure is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}—X—V_{H} or V_{H}—X—V_{L}, wherein X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form a Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

Single Chain Fv (scFv) Fragments

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favored linkers for a scFv.

In one example, the linker comprises the sequence SGGGGSGGGGSGGGGS.

The present disclosure also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present disclosure encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

### Half-Antibodies

In some examples, the antigen binding fragment of the present disclosure is a half-antibody or a half-molecule. The skilled artisan will be aware that a half antibody refers to a protein comprising a single heavy chain and a single light chain. The term “half antibody” also encompasses a protein comprising an antibody light chain and an antibody heavy chain, wherein the antibody heavy chain has been mutated to prevent association with another antibody heavy chain. In one example, a half antibody forms when an antibody dissociates to form two molecules each containing a single heavy chain and a single light chain.

Methods for generating half antibodies are known in the art and exemplary methods are described herein.

In one example, the half antibody can be secreted by introducing into cells genes of the single heavy chain and single light chain that constitute the IgG of interest for expression. In one example, a constant region (e.g., an IgG₄ constant region) comprises a “key or hole” (or “knob or hole”) mutation to prevent heterodimer formation. In one example, a constant region (e.g., an IgG₄ constant region) comprises a T366W mutation (or knob). In another example, a constant region (e.g., an IgG₄ constant region) comprises a T366S, L368A and Y407V mutation (or hole). In another example, the constant region comprises T350V, T366L, K392L and T394W mutations (knob). In another example, the constant region comprises T350V, L351Y, F405A and Y407V mutations (hole). Exemplary constant region amino acid substitutions are numbered according to the EU numbering system.

### Other Antibodies and Antibody Fragments

The present disclosure also contemplates other antibodies and antibody fragments, such as:

(i) minibodies, e.g., as described in U.S. Pat. No. 5,837,821;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab₃ (e.g., as described in EP19930302894).

### Stabilized Proteins

Binding proteins of the present disclosure can comprise an IgG4 constant region or a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione.

In one example, a stabilized IgG4 constant region comprises a proline at position 241 of the hinge region according to the system of Kabat (Kabat et al., Sequences of Proteins of Immunological Interest Washington D.C. United States Department of Health and Human Services, 1987 and/or 1991). This position corresponds to position 228 of the hinge region according to the EU numbering system (Kabat et al., Sequences of Proteins of Immunological Interest Washington D.C. United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010080538).

### Immunoglobulins and Immunoglobulin Fragments

An example of a binding protein of the present disclosure is a protein comprising a variable region of an immunoglobulin, such as a T cell receptor or a heavy chain immunoglobulin (e.g., an IgNAR, a camelid antibody).

Heavy Chain Immuno globulins

Heavy chain immunoglobulins differ structurally from many other forms of immunoglobulin (e.g., antibodies), in so far as they comprise a heavy chain, but do not comprise a light chain. Accordingly, these immunoglobulins are also referred to as “heavy chain only antibodies”. Heavy chain immunoglobulins are found in, for example, camelids and cartilaginous fish (also called IgNAR).

The variable regions present in naturally occurring heavy chain immunoglobulins are generally referred to as “V_{HH} domains” in camelid Ig and V-NAR in IgNAR, in order to distinguish them from the heavy chain variable regions that are present in conventional 4-chain antibodies (which are referred to as “V_{H} domains”) and from the light chain variable regions that are present in conventional 4-chain antibodies (which are referred to as “V_{L} domains”).

Heavy chain immunoglobulins do not require the presence of light chains to bind with high affinity and with high specificity to a relevant antigen. This means that single domain binding fragments can be derived from heavy chain immunoglobulins, which are easy to express and are generally stable and soluble.

A general description of heavy chain immunoglobulins from camelids and the variable regions thereof and methods for their production and/or isolation and/or use is found inter alia in the following references WO94/04678, WO97/49805 and WO 97/49805.

A general description of heavy chain immunoglobulins from cartilaginous fish and the variable regions thereof and methods for their production and/or isolation and/or use is found inter alia in WO2005118629.

### V-Like Proteins

In one example, a binding protein of the present disclosure comprises a T-cell receptor. T cell receptors have two V-domains that combine into a structure similar to the Fv module of an antibody. Novotny et al., Proc Natl Acad Sci USA 88: 8646-8650, 1991 describes how the two V-domains of the T-cell receptor (termed alpha and beta) can be fused and expressed as a single chain polypeptide and, further, how to alter surface residues to reduce the hydrophobicity directly analogous to an antibody scFv. Other publications describing production of single-chain T-cell receptors or multimeric T cell receptors comprising two V-alpha and V-beta domains include WO1999045110 or WO2011107595.

Other non-antibody proteins comprising antigen binding domains include proteins with V-like domains, which are generally monomeric. Examples of proteins comprising such V-like domains include CTLA-4, CD28 and ICOS. Further disclosure of proteins comprising such V-like domains is included in WO1999045110.

### Adnectins

In one example, a binding protein of the present disclosure comprises an adnectin. Adnectins are based on the tenth fibronectin type III (¹⁰Fn3) domain of human fibronectin in which the loop regions are altered to confer antigen binding. For example, three loops at one end of the β-sandwich of the ¹⁰Fn3 domain can be engineered to enable an Adnectin to specifically recognize an antigen. For further details see US20080139791 or WO2005056764.

### Anticalins

In a further example, a binding protein of the disclosure comprises an anticalin. Anticalins are derived from lipocalins, which are a family of extracellular proteins which transport small hydrophobic molecules such as steroids, bilins, retinoids and lipids. Lipocalins have a rigid β-sheet secondary structure with a plurality of loops at the open end of the conical structure which can be engineered to bind to an antigen. Such engineered lipocalins are known as anticalins. For further description of anticalins see U.S. Pat. No. 7,250,297 or US20070224633.

### Affibodies

In a further example, a binding protein of the disclosure comprises an affibody. An affibody is a scaffold derived from the Z domain (antigen binding domain) of Protein A of Staphylococcus aureus which can be engineered to bind to antigen. The Z domain consists of a three-helical bundle of approximately 58 amino acids. Libraries have been generated by randomization of surface residues. For further details see EP1641818.

### Avimers

In a further example, a binding protein of the disclosure comprises an Avimer. Avimers are multidomain proteins derived from the A-domain scaffold family. The native domains of approximately 35 amino acids adopt a defined disulphide bonded structure. Diversity is generated by shuffling of the natural variation exhibited by the family of A-domains. For further details see WO2002088171.

### DARPins

In a further example, a binding protein of the disclosure comprises a Designed Ankyrin Repeat Protein (DARPin). DARPins are derived from Ankyrin which is a family of proteins that mediate attachment of integral membrane proteins to the cytoskeleton. A single ankyrin repeat is a 33 residue motif consisting of two α-helices and a β-turn. They can be engineered to bind different target antigens by randomizing residues in the first α-helix and a β-turn of each repeat. Their binding interface can be increased by increasing the number of modules (a method of affinity maturation). For further details see US20040132028.

### Annexins

In one example, a binding protein of the present disclosure comprises an annexin.

Annexin, also known as lipocortin, form a family of soluble proteins that bind to membranes exposing negatively charged phospholipids, particularly phosphatidylserine (PS), in a Ca2+-dependent manner. Annexins are formed by a four- (exceptionally eight-) fold repeat of 70 amino-acid domains that are highly conserved and by a variable amino (N)-terminal domain, which is assumed to be responsible for their functional specificities. Annexins are important in various cellular and physiological processes such as providing a membrane scaffold, which is relevant to changes in the cell's shape. Annexins have also been shown to be involved in trafficking and organization of vesicles, exocytosis, endocytosis and also calcium ion channel formation

Annexin species II, V and XI are known to be located within the cellular membrane. Annexin A5 is the most abundant membrane-bound annexin scaffold. Annexin A5 can form 2-dimensional networks when bound to the phosphatidylserine unit of the membrane. Annexin A5 is effective in stabilizing changes in cell shape during endocytosis and exocytosis, as well as other cell membrane processes.

Annexin species I (or Annexin A1) is preferentially located on the cytosolic face of the plasma membrane and binds to the phosphatidylserine unit of the membrane. Annexin A1 does not form 2-dimensional networks on the activated membrane.

In one example, the annexin species is an annexin derivative or variant thereof. Annexin derivatives or variants thereof are known in the art and exemplary derivatives or variants are disclosed herein. By way of example, annexin variants/derivatives are disclosed in WO199219279, WO2002067857, WO2007069895, WO2010140886, WO2012126157, Schutters et al., Cell Death and Differentiation 20: 49-56, 2013, or Ungethüm et al., J Biol Chem., 286(3):1903-10, 2011.

For example, an annexin derivative may be truncated, e.g., include one or more domains or fewer amino acid residues than the native protein, or may contain substituted amino acids. In one example, the annexin derivative is a truncated Annexin 1. For example, the truncated Annexin 1 does not comprise the N-terminal self-cleavage site (e.g., 41 N-terminal amino acids have been deleted). In one example, a modified annexin may have an N-terminal chelation site comprising an amino acid extension, such as X₁-Gly-X₂ where X₁ and X₂ are selected from Gly and Cys. In one example, an annexin derivative or a modified annexin binds to phosphatidylserine. In one example, an annexin derivative or a modified annexin binds to phosphatidylserine at a similar level as the wildtype annexin. For example, an annexin derivative or modified annexin binds to phosphatidylserine at the same level as the wildtype annexin.

In one example, a membrane targeted binding protein of the present disclosure comprises a second binding region which is Annexin A5. In another example, a membrane targeted binding protein of the present disclosure comprises a second binding region which is Annexin A1. In one example, a membrane targeted binding protein of the present disclosure comprises an antibody or part thereof wherein each heavy chain is linked to an annexin that binds to a component on the plasma membrane. For example, the membrane targeted binding protein comprises a full-length antibody comprising two heavy chains each of which are linked to an annexin, such as Annexin A5 or Annexin A1. In another example, the membrane targeted binding protein of the present disclosure is a half antibody comprising a single heavy chain linked to a binding region comprising an annexin, such as Annexin A5 or Annexin A1. For the purposes of nomenclature only and not limitation, the amino acid sequence of an Annexin A5 is taught in Gene Accession ID 308, NCBI reference sequence NP_001145 and/or in SEQ ID NO: 14. In one example, the annexin has a sequence that is at least about 90% or 95% identical to an Annexin A5 sequence. In one example, the annexin is an annexin variant comprising a sequence set forth in SEQ ID NO: 26. For the purposes of nomenclature only and not limitation, the amino acid sequence of an Annexin A1 is taught in NCBI reference sequence NP_000691.1 and/or in SEQ ID NO: 29. In one example, the annexin has a sequence that is at least about 90% or 95% identical to an Annexin A1 sequence. In one example, the annexin in a truncated Annexin A1 sequence comprising a sequence set forth in SEQ ID NO: 30.

### Gamma-Carboxyglutamic Acid-Rich (GLA) Domains

In one example, the membrane targeted binding protein of the present disclosure comprises a gamma-carboxyglutamic acid-rich (GLA) domain or variant thereof.

The GLA domain contains glutamate residues that have been post-translationally modified by vitamin K-dependent carboxylation to form gamma-carboxyglutamate (Gla).

Proteins known to comprise a GLA domain are known in the art and include, but are not limited to, vitamin K-dependent proteins S and Z, prothrombin, transthyretin, osteocalcin, matrix GLA protein, inter-alpha-trypsin inhibitor heavy chain H2 and growth arrest-specific protein 6.

### Lactadherin Domains

In one example, the membrane targeted binding protein of the present disclosure comprises a lactadherin domain.

Lactadherin is a glycoprotein secreted by a variety of cell types and contains two EGF domains and two C domains (C1C2 and C2) with sequence homology to the C1 and C2 domains of blood coagulation factors V and VIII. Similar to these coagulation factors, lactadherin binds to phosphatidylserine (PS)-containing membranes with high affinity.

In one example, the lactadherin domain is a C1C2 domain (e.g., as set forth in SEQ ID NO: 27). In another example, the lactadherin domain is a C2 domain.

### Protein Kinase Domains

In one example, the present disclosure provides a membrane targeted binding protein comprising a protein kinase C domain.

Protein kinase C (PKC) is a family of protein kinase enzymes that are involved in controlling the function of other proteins through the phosphorylation of hydroxyl groups of serine and threonine amino acid residues on these proteins, or a member of this family.

The structure of PKC is known in the art and consists of a regulatory domain and a catalytic domain tethered together by a hinge region. The regulatory domain comprises a C1 and a C2 domain which bind to DAG and Ca²⁺ respectively to recruit PKC to the plasma membrane.

In one example, the protein kinase C domain is the C1 domain. In another example, the protein kinase C domain is the C2 domain.

### Pleckstrin Homology Domain

In one example, the present disclosure provides a membrane targeted binding protein comprising a pleckstrin homology (PH) domain.

The PH domain is known in the art and is a small modular domain that occurs in a wide range of proteins involved in intracellular signalling or as a constituent of the cytoskeleton. The PH domain comprises approximately 120 amino acids. The domains can bind phosphatidylinositol within biological membranes and proteins such as the beta/gamma subunits of heterotrimeric G proteins. Through these interactions, PH domains play a role in recruiting proteins to different membranes, thus targeting them to appropriate cellular compartments or enabling them to interact with other components of the signal transduction pathways.

### Phosphatidylserine-Interacting Peptides

In one example, the present disclosure provides a membrane targeted binding protein comprising a phosphatidylserine-interacting peptide to target the membrane component. Suitable peptides are known in the art and include, for example, PSP1 as described in Thapa et al., J. Cell. Mol. Med. 12. 1649-1660, 2008 and Kim et al., PLOS One, 10(3): e0121171. PSP1 comprises the sequence CLSYYPSYC (SEQ ID NO: 28). The present disclosure also contemplates variants of PSP1 that retain its ability to bind phosphatidylserine.

In one example, a membrane targeted binding protein of the present disclosure comprises an antibody or part thereof wherein each heavy chain (or light chain) is linked to PSP1 or a variant thereof that binds to a component on the plasma membrane. For example, the membrane targeted binding protein comprises a full-length antibody comprising two heavy chains (or two light chains) each of which are linked to PSP1. In another example, the membrane targeted binding protein of the present disclosure is a half antibody comprising a single heavy chain (or light chain) linked to a binding region comprising PSP1.

### Linkers

In one example, the first binding region of the membrane targeted binding protein is linked to the second binding region via a linker. For example, the linker is a linker peptide.

In one example, an intervening peptidic linker may be introduced between the first and second binding region.

In one example, the linker is a flexible linker. For example, the linker joins the N-terminus of the second binding region to the N- or C-terminus of a heavy chain or domain thereof or a light chain or domain thereof of the anti-Factor IX antibody or antigen binding fragment thereof.

A “flexible” linker is an amino acid sequence which does not have a fixed structure (secondary or tertiary structure) in solution. Such a flexible linker is therefore free to adopt a variety of conformations. Flexible linkers suitable for use in the present disclosure are known in the art. An example of a flexible linker for use in the present invention is the linker sequence SGGGGS/GGGGS/GGGGS or (Gly₄Ser)₃. Flexible linkers are also disclosed in WO1999045132.

The linker may comprise any amino acid sequence that does not substantially hinder interaction of the binding region with its target. Preferred amino acid residues for flexible linker sequences include, but are not limited to, glycine, alanine, serine, threonine proline, lysine, arginine, glutamine and glutamic acid.

The linker sequences between the binding regions preferably comprise five or more amino acid residues. The flexible linker sequences according to the present disclosure consist of 5 or more residues, preferably, 5, 6, 7, 8, 9, 10, 11, 12, 13, 14, 15, 16, 17, 18, 19 or 20 or more residues. In a highly preferred embodiment of the invention, the flexible linker sequences consist of 5, 7, 10 or 16 residues.

In one example, the flexible linker has an amino acid sequence according to SEQ ID NO: 20, i.e., SGGGGSGGGGSGGGGS (GS16).

In another example, the linker has the amino acid sequence SG (GS2).

In another example, the linker has the amino acid sequence according to SEQ ID NO: 24, i.e., SGGGGS (GS6).

In a further example, the linker has the amino acid sequence according to SEQ ID NO: 25, i.e., SGGGGSGGGGSGGGGSGGGGSGGGGSGGGGS (GS31).

In one example, the linker is a rigid linker. A “rigid linker” (including a “semi-rigid linker”) refers to a linker having limited flexibility. For example, the relatively rigid linker comprises the sequence (EAAAK)ₙ, where n is between 1 and 3. The value of n can be between 1 and about 10 or between about 1 and 100. For example, n is at least 1, or at least 2, or at least 3, or at least 4, or at least 5, or at least 6, or at least 7, or at least 8, or at least 9, or at least 10. In one example, n is less than 100. For example, n is less than 90, or less than about 80, or less than about 60, or less than about 50, or less than about 40, or less than about 30, or less than about 20, or less than about 10. A rigid linker need not completely lack flexibility.

In one example, the linker is a cleavable linker. For example, the linker comprises a cleavage site for a peptidase. For example, the linker comprises a cleavage site for urokinase, pro-urokinase, plasmin, plasminogen, TGFβ, staphylokinase, Thrombin, a coagulation factor (e.g., Factor IXa, Factor Xa) or a metalloproteinase, such as an interstitial collagenase, a gelatinase or a stromelysin. Exemplary cleavable linkers are described in U.S. Pat. Nos. 6,004,555, 5,877,289, 6,093,399 and 5,877,289.

### Plasma Membrane Targets

The present disclosure provides a membrane targeted binding protein that specifically binds to a component of a plasma membrane of a mammalian cell. For example, the membrane targeted binding protein binds to a component via a binding region. Binding of the binding protein to the component of a plasma membrane of a mammalian cell enhances the activity of the binding protein.

Plasma membrane targets are known in the art. Exemplary plasma membrane targets include, but are not limited to, aminophospholipids, and membrane-associated polypeptides.

In one example, the membrane-associated polypeptide is not a coagulation factor. In one example, the membrane-associated polypeptide is not factor X/Xa. In one example, the membrane-associated polypeptide is not factor X/Xa if the first binding region binds to factor IX/IXa.

### Aminophospholipids

In one example, the present disclosure provides a membrane targeted binding protein that specifically binds an aminophospholipid on the plasma membrane of a mammalian cell.

The term “aminophospholipids” encompasses any phospholipid that contains one or more amino groups. Aminophospholipids are located on the inner surface of the plasma membrane of healthy mammalian cells. However, during cell aging, apoptosis and immune cell activation, aminophospholipids translocate to the outer surface of the plasma membrane. Translocation of aminophospholipids to the outer surface of the plasma membrane aids coagulation factor binding.

Exemplary aminophospholipids are known in the art. For example, the aminophospholipid is a phosphatidylserine or a phosphatidylethanolamine.

Compounds that bind to aminophospholipids are known in the art and exemplary compounds are described herein. For example compounds that bind aminophospholipids include annexins, such as Annexin A5 as discussed above.

### Membrane Associated Polypeptides

In one example, the present disclosure provides a membrane targeted binding protein that specifically binds a membrane associated polypeptide on the plasma membrane of a mammalian cell.

Exemplary membrane associated polypeptides are known in the art and include, for example, glycoprotein IIb/IIIa (GPIIb/IIIa), beta-2 glycoprotein 1 (β2GP1), transcript-1 (TLT-1), a coagulation factor and a selectin.

### Glycoprotein IIb/IIIa (GPIIb/IIIa)

Glycoprotein IIb/IIIa is an integrin complex found on platelets. Typically, it is a receptor for fibrinogen and von Willebrand factor and aids in platelet activation. The complex is formed via calcium-dependent association of gpIIb and gpIIIa, a required step in normal platelet aggregation and endothelial adherence.

In one example, the present disclosure provides a membrane targeted binding protein that specifically binds a GPIIb/IIIa on the plasma membrane of a mammalian cell.

Compounds that bind to glycoprotein IIb/IIIa are known in the art and exemplary compounds are described herein. For example, glycoprotein IIb/IIIa antagonists are commercially available and include Abciximab (RePro®), Eptifibatid (Integrilin®) and Tirofiban (Aggrastat®).

### Beta-2 Glycoprotein 1 (β2GP1)

Beta-2 glycoprotein 1 (also known as apolipoprotein H or Apo-H) is a 38 kDa multifunctional apolipoprotein that in humans is encoded by the APOH gene. β2GP1 is involved in agglutination of platelets by inhibition of serotonin release by platelets. Apo-H is synthesized by hepatocytes, endothelial cells and trophoblast cells.

In one example, the present disclosure provides a membrane targeted binding protein that specifically binds a βGP1 on the plasma membrane of a mammalian cell.

### TREM-Like Transcript-1 (TLT-1)

TLT-1 is a membrane bound protein receptor belonging to the TREM family of proteins. TLT-1 is found in alpha-granules of platelets and megakaryocytes. Upon platelet activation TLT-1 is rapidly brought to the surface of platelets.

In one example, the present disclosure provides a membrane targeted binding protein that specifically binds a TLT-1 on the plasma membrane of a mammalian cell.

Compounds that bind to TREM-like transcript-1 are known in the art and described, for example, in U.S. Pat. No. 7,553,936.

### Selectins

Selectins (cluster of differentiation 62 or CD62) are a family of cell adhesion molecules (or CAMs). All selectins are single-chain transmembrane glycoproteins that share similar properties to C-type lectins due to a related amino terminus and calcium-dependent binding. Selectins bind to sugar moieties and so are considered to be a type of lectin, cell adhesion proteins that bind sugar polymers.

All three known members of the selectin family (L-, E-, and P-selectin) share a similar cassette structure: an N-terminal, calcium-dependent lectin domain, an epidermal growth factor (EGF)-like domain, a variable number of consensus repeat units (2, 6, and 9 for L-, E-, and P-selectin, respectively), a transmembrane domain (TM) and an intracellular cytoplasmic tail (cyto).

L-selectin is the smallest of the vascular selectins, expressed on all granulocytes and monocytes and on most lymphocytes, can be found in most leukocytes. P-selectin, the largest selectin, is stored in α-granules of platelets and in Weibel-Palade bodies of endothelial cells, and is translocated to the cell surface of activated endothelial cells and platelets. E-selectin is not expressed under baseline conditions, except in skin microvessels, but is rapidly induced by inflammatory cytokines.

Compounds that bind to selectins are known in the art and described, for example, in U.S. Pat. No. 5,800,815 (P-selectins), U.S. Pat. No. 5,632,991 (E-selectins) and U.S. Pat. No. 5,756,095 (E- and L-selectins).

In one example, the present disclosure provides a membrane targeted binding protein that specifically binds a selectin on the plasma membrane of a mammalian cell. For example, the selectin is a P-selectin.

### Screening Assays

Suitable methods for selecting a membrane targeted binding protein that specifically binds to at least one blood coagulation factor are available to those skilled in the art. For example, a screen may be conducted to identify binding proteins capable of binding to coagulation factors.

Similarly, amounts and timing of administration of a membrane targeted binding protein suitable for use in a method described herein can be determined or estimated using techniques known in the art, e.g., as described below.

### Blood Coagulation Assay

The present disclosure provides membrane targeted binding proteins that comprise a binding region that binds a coagulation factor. To determine the coagulation activity of the membrane targeted binding protein an in vitro assay can be used.

In one example, the coagulation activity is indicative of the bypassing activity of the membrane targeted binding protein.

In one example, the coagulation activity of the membrane targeted binding protein can be measured based on the activated partial thromboplastin time (aPTT). For example, factor deficient plasma is incubated with phospholipid, a contact activator, and varying concentrations of the membrane targeted coagulation factor binding protein followed by calcium. Addition of calcium initiates coagulation and timing begins. The aPTT is the time taken for a fibrin clot to form.

aPTT can be determined using standard methodology or assays known in the art, e.g., Thrombolyzer Compact X system (Behnk Elektronik).

Membrane targeted binding proteins that are found to effectively enhance coagulation activity (i.e., induce a fibrin clot) are identified as membrane targeted binding proteins of the present disclosure.

### Chromogenic FVIII Assay

For membrane targeted binding proteins that comprise a binding region that binds factor IX, the factor VIII bypassing activity can be measured using a chromogenic factor VIII assay.

In one example, assay buffer is pre-mixed with factor IXa, factor X and phospholipids. The membrane targeted binding protein of the present disclosure is added followed by calcium and chromogenic substrate. Following cessation of the chromogenic reaction, the factor VIII bypassing activity of the binding protein is assessed.

Chromogenic assays for measuring factor VIII activity and/or FVIII bypassing activity are known in the art and include, for example, COATEST SP4 FVIII (Chromogeneix).

Membrane targeted binding proteins that are found to demonstrate FVIII bypassing activity are identified as binding proteins of the present disclosure.

### Determining Affinity

Optionally, the dissociation constant (Kd) or association constant (Ka) or equilibrium constant (K_{D}) of a binding region for a coagulation factor is determined. These constants for a binding region (e.g., an antibody or antigen binding fragment) are, in one example, measured by biosensor analysis using surface plasmon resonance assays, with immobilized phosphatidylserine containing vesicles. Exemplary SPR methods are described in U.S. Pat. No. 7,229,619.

Affinity measurements can be determined by standard methodology for antibody reactions, for example, immunoassays, surface plasmon resonance (SPR) (Rich and Myszka Curr. Opin. Biotechnol 11: 54, 2000; Englebienne Analyst. 123: 1599, 1998), isothermal titration calorimetry (ITC) or other kinetic interaction assays known in the art.

### In Vitro Cellular Assays

In one example, the uptake and recycling of the membrane targeted binding protein is tested in an in vitro cellular assay.

Methods of assessing cellular uptake and recycling are known in the art and/or exemplified herein. For example, fluorescently labelled membrane targeted binding protein is incubated with cells expressing the human FcRn receptor on the cell surface. After addition of the labelled membrane targeted binding protein the progress of the binding protein recycling can be tracked and compared to non-targeted binding protein by confocal fluorescence microscopy. Changes to the normal recycling pathway for a particular membrane targeted binding protein can be identified and characterised.

Membrane targeted binding proteins that are found to be effectively recycled are identified as binding proteins of the present disclosure.

### Thrombin Generation Assays

In one example, the activation of the intrinsic and/or extrinsic coagulation pathway is assessed in a thrombin generation assay.

Methods of assessing activation of the intrinsic and/or extrinsic coagulation pathway are known in the art (e.g., Thrombinoscope) and/or exemplified herein. For example, the membrane targeted binding protein is mixed with an activator of the intrinsic or extrinsic pathway, tissue factor and phospholipids. Following addition of a Fluo-substrate, thrombin generation is monitored and calculated.

Membrane targeted binding proteins that are found to effectively enhance the intrinsic and/or extrinsic coagulation pathway are identified as membrane targeted binding proteins of the present disclosure.

### Pharmacokinetic Analysis

In one example, the pharmacokinetic (PK) properties of the membrane targeted binding protein will be assessed.

Methods of assessing the PK properties are known in the art and/or are exemplified herein. For example, membrane targeted binding proteins are injected into transgenic mice expressing human FcRn receptor. Plasma levels of membrane targeted binding protein will be assessed using ELISA using commercially available antibodies.

### In Vivo Animal Models

In one example, the method of treating a bleeding disorder with a membrane targeted binding protein is tested in an animal model of a bleeding disorder.

Animal models of bleeding disorders are known in the art. A membrane targeted binding protein can be administered to such an animal model.

In one example, the animal model is a model of haemophilia, for example, haemophilia A. For example, the mouse model is a FVIII knockout mouse model such as that described in Bi L. et al., 1995 Targeted disruption of the mouse factor VIII gene produces a model of haemophilia A. Nature Genetics 10(1):119-21. The effect of the membrane targeted binding protein on coagulation in such a mouse is determined, e.g. in a tail clip assay.

In one example, human factor IX and/or human factor X are administered to the FVIII deficient mouse. The effect of the membrane targeted binding protein on coagulation in such a treated FVIII deficient mouse is determined, e.g. in a tail clip assay.

### Bethesda Assay

In one example, the development of inhibitors against a membrane targeted binding protein can be determined using an in vitro coagulation assay, e.g., using commercially available kits, such as the Bethesda assay (Affinity Biologicals) and/or FVIII Inhibitor Reagent Kit (Technoclone).

### Pharmaceutical Compositions

Suitably, in compositions or methods for administration of the membrane targeted binding protein of the disclosure to a subject, the membrane targeted binding protein is combined with a pharmaceutically acceptable carrier as is understood in the art. Accordingly, one example of the present disclosure provides a composition (e.g., a pharmaceutical composition) comprising the membrane targeted binding protein of the disclosure combined with a pharmaceutically acceptable carrier.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for example described in Remington's Pharmaceutical Sciences (Mack Publishing Co. N.J. USA, 1991).

A membrane targeted binding protein that binds at least one blood coagulation factor is useful for parenteral, topical, oral, or local administration, aerosol administration, or transdermal administration, for prophylactic or for therapeutic treatment. In one example, the membrane targeted binding protein is administered parenterally, such as subcutaneously or intravenously. For example, the membrane targeted binding protein administered intravenously.

Formulation of a membrane targeted binding protein to be administered will vary according to the route of administration and formulation (e.g., solution, emulsion, capsule) selected. An appropriate pharmaceutical composition comprising a membrane targeted binding protein to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The membrane targeted binding protein can be stored in the liquid stage or can be lyophilized for storage and reconstituted in a suitable carrier prior to use according to art-known lyophilization and reconstitution techniques.

### Methods of Treating or Preventing Bleeding Disorders

As discussed herein, the present disclosure provides a method of treating or preventing a disease or condition in a subject, the method comprising administering the membrane targeted binding protein of the present disclosure or the composition of the present disclosure to a subject in need thereof. In one example, the present disclosure provides a method of treating a disease or condition in a subject in need thereof.

The present disclosure also provides for use of a membrane targeted binding protein of the present disclosure for treating or preventing a disease or condition in a subject comprising administering the membrane targeted binding protein of the present disclosure or the composition of the present disclosure to a subject in need thereof. In one example, the present disclosure provides for use of a membrane targeted binding protein of the present disclosure for treating a disease or condition in a subject in need thereof.

In one example, the disease or condition is a bleeding disorder.

In one example, the subject suffers from a bleeding disorder. The bleeding disorder can be inherited or acquired. For example, a subject suffering from a bleeding disorder has suffered a symptom of a bleeding disorder, such as:
Easy bruising;
Bleeding gums;
Heavy bleeding from small cuts or dental work;
Unexplained nosebleeds;
Heavy menstrual bleeding;
Bleeding into joints; and/or
Excessive bleeding following surgery.

In one example, the subject is at risk of developing a bleeding disorder. A subject is at risk if he or she has a higher risk of developing a bleeding disorder than a control population. The control population may include one or more subjects selected at random from the general population (e.g., matched by age, gender, race and/or ethnicity) who have not suffered from or have a family history of angina, stroke and/or heart attack. A subject can be considered at risk for a bleeding disorder if a “risk factor” associated with a bleeding disorder is found to be associated with that subject. A risk factor can include any activity, trait, event or property associated with a given disorder, for example, through statistical or epidemiological studies on a population of subjects. A subject can thus be classified as being at risk for a bleeding disorder even if studies identifying the underlying risk factors did not include the subject specifically. For example, a subject who has excessive bleeding is at risk of developing a bleeding disorder because the frequency of a bleeding disorder is increased in a population of subjects who have excessive bleeding as compared to a population of subjects who do not.

In one example, the subject is at risk of developing a bleeding disorder and the membrane targeted binding protein is administered before or after the onset of symptoms of a bleeding disorder. In one example, the membrane targeted binding protein is administered before the onset of symptoms of a bleeding disorder. In one example, the membrane targeted binding protein is administered after the onset of symptoms of a bleeding disorder. In one example, the membrane targeted binding protein of the present disclosure is administered at a dose that alleviates or reduces one or more of the symptoms of a bleeding disorder in a subject at risk.

The methods of the present disclosure can be readily applied to any form of bleeding disorder in a subject.

A method of the present disclosure may also include co-administration of the at least one membrane targeted binding protein according to the disclosure together with the administration of another therapeutically effective agent for the prevention or treatment of a bleeding disorder.

In one example, the membrane targeted binding protein of the disclosure is used in combination with at least one additional known compound or therapeutic protein which is currently being used or is in development for preventing or treating bleeding disorders. Compounds currently used in the treatment of bleeding disorders are known in the art. Exemplary therapeutic proteins may be plasma derived from a donor or a recombinant protein. For example, the therapeutic protein is a plasma derived or recombinant coagulation factor protein. For example, the therapeutic protein is selected from the group consisting of factor I, factor II ((prothrombin)/thrombin), factor III, factor V, factor VII, factor VIIa (e.g., NovoSeven®), factor VIII (such as a single chain recombinant factor VIII, e.g., as described in Zollner et al., Thromb Res. 132:280-287, 2013; or a plasma derived factor VIII product, such as FEIBA®, Monoclate-P®, or Biostate®; or a recombinant factor VIII product, such as Advate®, Eloctate®, Recombinate®, Kogenate Fs®, Helixate® Fs, Helixate®, Xyntha®/Refacto Ab®, Hemofil-M®, Monarc-M®, Alphanate®, Koate-Dvi®, Nuwiq® or Hyate:C®), factor IX (e.g., a plasma derived factor IX product such as, Berinin® P, MonoFIX® or Mononine®; or a recombinant factor IX product such as Alphanine SD®, Alprolix®, Bebulin®, Bebulin VH®, Benefix®, Ixinity®, Profilnine SD®, Proplex T®, or Rixubis®), factor X, factor XI, factor XII and factor XIII (e.g., Fibrogammin® P, Corifact®, Cluvot® or Cluviat®). In one example, the therapeutic protein is a von Willebrand factor/FVIII complex (e.g., Humate-P®, Haemate®-P, Biostate® or Voncento®). In an alternative example, the therapeutic protein is a prothrombin complex (e.g., Beriplex® P/N, Confidex® or Kcentra®). In another example, the therapeutic protein is a fibrinogen (e.g., RiaSTAP®, Haemocomplettan® P). In one example, the therapeutic protein is a modified form of a coagulation factor, e.g., as described herein.

As will be apparent from the foregoing, the present disclosure provides methods of concomitant therapeutic treatment of a subject, comprising administering to a subject in need thereof an effective amount of a first agent and a second agent, wherein the first agent is a membrane targeted binding protein of the present disclosure, and the second agent is also for the prevention or treatment of a bleeding disorder.

As used herein, the term “concomitant” as in the phrase “concomitant therapeutic treatment” includes administering a first agent in the presence of a second agent. A concomitant therapeutic treatment method includes methods in which the first, second, third or additional agents are co-administered. A concomitant therapeutic treatment method also includes methods in which the first or additional agents are administered in the presence of a second or additional agent, wherein the second or additional agent, for example, may have been previously administered. A concomitant therapeutic treatment method may be executed step-wise by different actors. For example, one actor may administer to a subject a first agent and as a second actor may administer to the subject a second agent and the administering steps may be executed at the same time, or nearly the same time, or at distant times, so long as the first agent (and/or additional agents) are after administration in the presence of the second agent (and/or additional agents). The actor and the subject may be the same entity (e.g. a human).

The optimum concentration of the active ingredient(s) in the chosen medium can be determined empirically, according to procedures known to the skilled artisan, and will depend on the ultimate pharmaceutical formulation desired.

The dosage ranges for the administration of the binding protein of the disclosure are those large enough to produce the desired effect. For example, the composition comprises an effective amount of the membrane targeted binding protein. In one example, the composition comprises a therapeutically effective amount of the membrane targeted binding protein. In another example, the composition comprises a prophylactically effective amount of the membrane targeted binding protein.

The dosage should not be so large as to cause adverse side effects, such as paradoxical bleedings and development of inhibitors. Generally, the dosage will vary with the age, condition, sex and extent of the disease in the patient and can be determined by one of skill in the art. The dosage can be adjusted by the individual physician in the event of any complication.

Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

In some examples, the membrane targeted binding protein is administered at an initial (or loading) dose which is higher than subsequent (maintenance doses). For example, the membrane targeted binding protein is administered at an initial dose of between about 10 mg/kg to about 30 mg/kg. The binding protein is then administered at a maintenance dose of between about 0.0001 mg/kg to about 10 mg/kg. The maintenance doses may be administered every 7-35 days, such as, every 7 or 14 or 28 days.

In some examples, a dose escalation regime is used, in which a membrane targeted binding protein is initially administered at a lower dose than used in subsequent doses. This dosage regime is useful in the case of subject's initially suffering adverse events In the case of a subject that is not adequately responding to treatment, multiple doses in a week may be administered. Alternatively, or in addition, increasing doses may be administered.

A subject may be retreated with the membrane targeted binding protein, by being given more than one exposure or set of doses, such as at least about two exposures of the binding protein, for example, from about 2 to 60 exposures, and more particularly about 2 to 40 exposures, most particularly, about 2 to 20 exposures.

In one example, any retreatment may be given when signs or symptoms of disease return, e.g., a bleeding episode.

In another example, any retreatment may be given at defined intervals. For example, subsequent exposures may be administered at various intervals, such as, for example, about 24-28 weeks or 48-56 weeks or longer. For example, such exposures are administered at intervals each of about 24-26 weeks or about 38-42 weeks, or about 50-54 weeks.

In the case of a subject that is not adequately responding to treatment, multiple doses in a week may be administered. Alternatively, or in addition, increasing doses may be administered.

In another example, for subjects experiencing an adverse reaction, the initial (or loading) dose may be split over numerous days in one week or over numerous consecutive days.

Administration of a membrane targeted binding protein according to the methods of the present disclosure can be continuous or intermittent, depending, for example, on the recipient's physiological condition, whether the purpose of the administration is therapeutic or prophylactic, and other factors known to skilled practitioners. The administration of an agent may be essentially continuous over a preselected period of time or may be in a series of spaced doses, e.g., either during or after development of a condition.

### Kits and Other Compositions of Matter

Another example of the disclosure provides kits containing a membrane targeted binding protein of the present disclosure useful for the treatment or prevention of a bleeding disorder as described above.

In one example, the kit comprises (a) a container comprising a membrane targeted binding protein optionally in a pharmaceutically acceptable carrier or diluent; and (b) a package insert with instructions for treating or preventing a bleeding disorder in a subject.

In one example, the kit comprises (a) at least one membrane targeted binding protein that binds to a blood coagulation factor; (b) instructions for using the kit in treating or preventing the bleeding disorder in the subject; and (c) optionally, at least one further therapeutically active compound or drug.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for treating atherosclerosis and may have a sterile access port (for example, the container may be an intravenous solution bag or a vial having a stopper pierceable by a hypodermic injection needle). At least one active agent in the composition is the membrane targeted binding protein. The label or package insert indicates that the composition is used for treating a subject eligible for treatment, e.g., one having or predisposed to developing a bleeding disorder, with specific guidance regarding dosing amounts and intervals of binding protein and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

The kit optionally further comprises a container comprises a second medicament, wherein the membrane targeted binding protein is a first medicament, and which article further comprises instructions on the package insert for treating the subject with the second medicament, in an effective amount. The second medicament may be a therapeutic protein set forth above.

The present disclosure includes the following non-limiting Examples.

### EXAMPLES

### Example 1: Generation and Purification of Recombinant Antibodies

Expression constructs were generated using standard molecular biology methods. Nucleotide sequences encoding the antibodies, Annexin A5 (NP_001145; SEQ ID NO: 14) and GS linker (SEQ ID NO: 20) were synthesized by Geneart (Thermo Fisher Scientific, NY, USA). Sequences were amplified by PCR, digested by restriction and cloned into expression vector by T4 DNA ligase.

Antibodies were generated according to Table 1 below.

TABLE 1
Recombinant antibodies

| Antibody | SEQ ID NO | Identifier |
|---|---|---|
| Anti-Factor IX/X bispecific | Light chain X - SEQ ID NO: 1 | CSL3415/3416 |
| antibody | Heavy chain X - SEQ ID NO: 2 | |
| | Light chain IX - SEQ ID NO: 1 | |
| | Heavy chain IX - SEQ ID NO: 3 | |
| Anti-Factor X monospecific | Light chain - SEQ ID NO: 1 | CSL3491 |
| antibody | Heavy chain - SEQ ID NO: 4 | |
| Anti-FIX monospecific antibody | Light chain - SEQ ID NO: 1 | CSL3492 |
| | Heavy chain - DEQ ID NO: 5 | |
| Anti-FIX monospecific half | Light chain - SEQ ID NO: 1 | CSL3535 |
| antibody | Heavy chain - SEQ ID NO: 6 | |
| Anti-FX monospecific half | Light chain - SEQ ID NO: 1 | CSL3572 |
| antibody | Heavy chain - SEQ ID NO: 7 | |
| Annexin A5 linked | Light chain - SEQ ID NO: 1 | CSL4060 |
| monospecific anti-FIX antibody | Heavy chain - SEQ ID NO: 8 | |
| Annexin A5 linked anti-FIX | Light chain - SEQ ID NO: 1 | CSL4062 |
| half antibody | Heavy chain - SEQ ID NO: 9 | |
| Annexin A5 linked anti-FX half | Light chain - SEQ ID NO: 1 | CSL4063 |
| antibody | Heavy chain - SEQ ID NO: 10 | |
| Annexin A5 linked anti-FIX/FX | Light chain - SEQ ID NO: 1 | CSL4062/4063 |
| bispecific antibody | FIX Heavy chain - SEQ ID NO: 9 | |
| | FX heavy chain - SEQ ID NO: 10 | |
| Annexin A5 linked (single A5 | Light chain - SEQ ID NO: 1 | CSL3535/4063 |
| molecule) anti-FIX/FX | FIX Heavy chain - SEQ ID NO: 6 | |
| bispecific antibody | FX Heavy chain - SEQ ID NO: 10 | |
| Annexin A5 linked (single A5 | Light chain - SEQ ID NO: 1 | CSL3572/4062 |
| molecule) anti-FIX/FX | FIX Heavy chain - SEQ ID NO: 9 | |
| bispecific antibody | FX Heavy chain - SEQ ID NO: 7 | |

Recombinant plasmid DNA was purified using the QIAprep Spin Miniprep kit (QIAGEN, Hilden, Germany) and quantified by Nanodrop UV spectrophotometer. Sequences were confirmed prior to transfection.

All transfections were performed by transient transfection using the Expi293F™ Expression System according to the manufacturer's instructions.

Protein was harvested from the resultant conditioned media clarified by centrifugation and filtration prior to purification. Small scale (mg) robotic antibody purification was performed as previously described (Schmidt et al. Journal of Chromatography, 1455 (2016) 9-19).

### Example 2: Annexin A5 Linked Antibodies are Membrane Targeted

To assess whether Annexin A5 linked antibodies were targeted to the cellular membrane, biosensor analysis of Annexin A5 linked antibodies was performed. Briefly, phosphatidylserine (PS)/phosphatidylcholine (PC)/phosphorylethanolamine (PE) (PS/PC/PE-biotinyl 70:25:5) phospholipid mixtures were solubilized in TRIS [20 mM] pH 8.0, NaCl [150 mM], NOG [2 mM] and vesicles obtained using sonication. Phospholipids lacking phosphatidylserine (PC/PE-biotinyl 95:5) were prepared in a similar fashion to act as a reference surface in biosensor studies.

Phosphatidylserine containing vesicles were immobilised at low levels on an active flow cell of a Biacore® T-200 biosensor docked with a SA sensor chip. Vesicles lacking PS were immobilised on an upstream reference cell. Binding to PS/PC/PE was assessed at 37° C. by injecting 3.3, 1.1, 0.37, 0.12 and 0.04 nM aFIX-Annexin A5 (CSL4060) for 5 minutes. Responses at the end of the binding phase were used to fit the data to a 1:1 steady-state binding model to determine the affinity of the interaction (KD). Running buffer used throughout was HEPES [10 mM] pH 7.3, NaCl [150 mM], CaCl2 [2 mM] with 0.1% BSA.

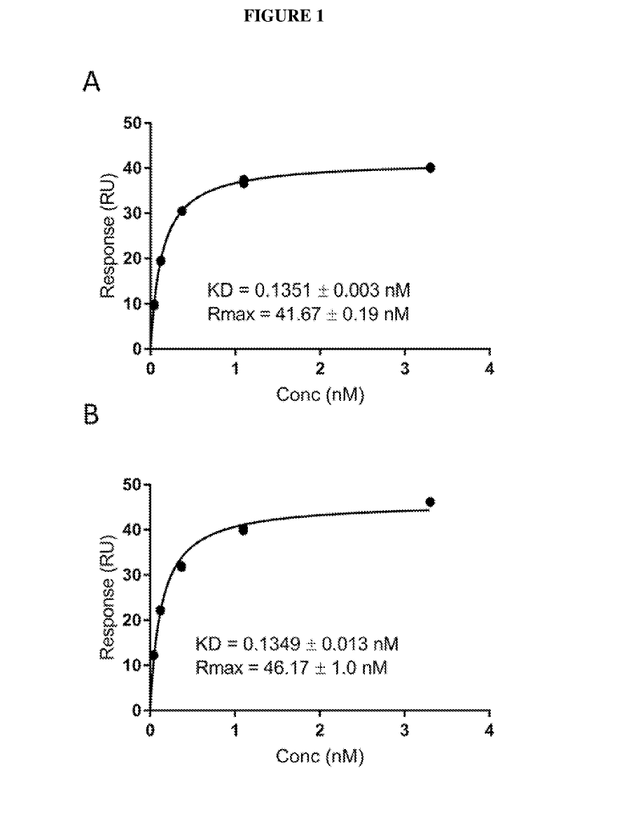

FIG. 1A illustrates that a monospecific anti-Factor IX-Annexin A5 linked antibody (CSL4060), which comprises two Annexin A5 molecules, bound to phosphatidylserine containing vesicles with an affinity of 0.1351±0.003 nM and an Rmax of 41.67±0.19 nM. Additionally, FIG. 1B shows that a bispecific anti-Factor IX/X-Annexin A5 linked antibody (CSL4062/3572), comprising one Annexin A5 molecule, bound to phosphatidylserine containing vesicles with an affinity of 0.1349±0.013 nM and an Rmax of 46.17±1.0 nM. Each experiment was repeated twice.

### Example 3: Membrane Targeted Anti-Factor IX Monospecific Full and Half Antibodies have Increased Factor VIII Bypassing Activity Compared to Non-Targeted Antibodies

To investigate the potential Factor VIII-bypassing activity and coagulation activity of the generated antibodies the Activated Partial Thromboplastin Time (aPTT) was measured using a Thrombolyzer Compact X system (Behnk Elektronik, Germany) with standard assay reagents from Siemens Healthcare (Siemens, Germany) as per the manufacturer's instructions. The antibodies were diluted in FVIII-deficient plasma (Siemens Healthcare) to achieve final concentrations between 1000 nM and 1 pM as indicated in the figures. Briefly, 50 ul of each dilution was mixed with 50 ul of aPTT reagent (Pathromtin SL) in one side of the Thrombolyzer cuvettes. 50 ul CaCl₂ [25 mM] was added to the other side of the Thrombolyzer cuvettes and temperature was allowed equilibrate to 37° C. The coagulation reaction was initiated by mixing the CaCl₂ solution with the antibody/aPTT reagent mix and coagulation was continuously monitored at 405 nm and 620 nm wavelength. Time to clot formation was graphed against the antibody concentration. To fit values to a curve and permit calculation of EC₅₀ values, the highest concentration of the Annexin A5-targeted aFIX antibody was omitted and the lower end of the curve was set to 24 sec (similar to the coagulation time observed with non-membrane-targeted anti-Factor IX/X bispecific antibody CSL3415/3416 at 1000 nM).

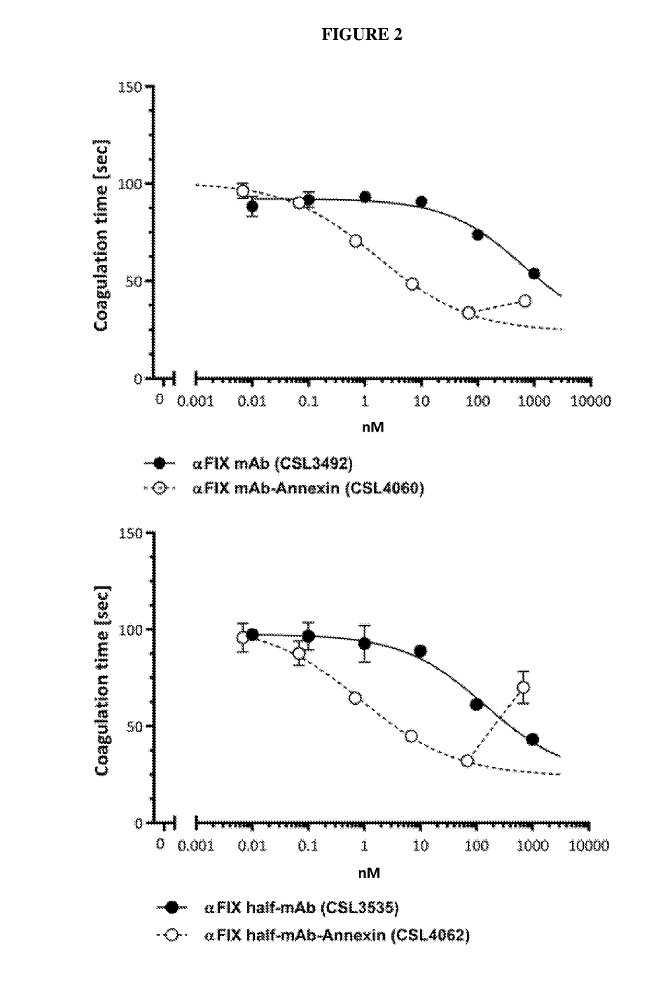

As shown in FIG. 2, the membrane-targeted full and half anti-Factor IX monospecific antibodies have increased Factor VIII bypassing activity compared to the non-targeted antibodies. FIG. 2A shows results of three or four independent experiments demonstrating that the membrane targeted full anti-Factor IX antibody (CSL4060) has increased bypassing activity (EC₅₀ 1.64 nM) compared to the non-membrane targeted full anti-Factor IX antibody CSL3492 (EC₅₀ 621 nM). Additionally, FIG. 2B shows results of three independent experiments demonstrating that the membrane targeted half anti-Factor IX antibody (CSL4062) has increased bypassing activity (EC₅₀ 0.89 nM) compared to the non-membrane targeted half anti-Factor IX antibody CSL3535 (EC₅₀ 142 nM).

### Example 4: Membrane Targeted Anti-Factor IX/X Bispecific Antibodies have Increased Factor VIII Bypassing Activity Compared to Non-Targeted Antibodies

Factor VIII bypassing activity was measured using the aPTT assay as described above.

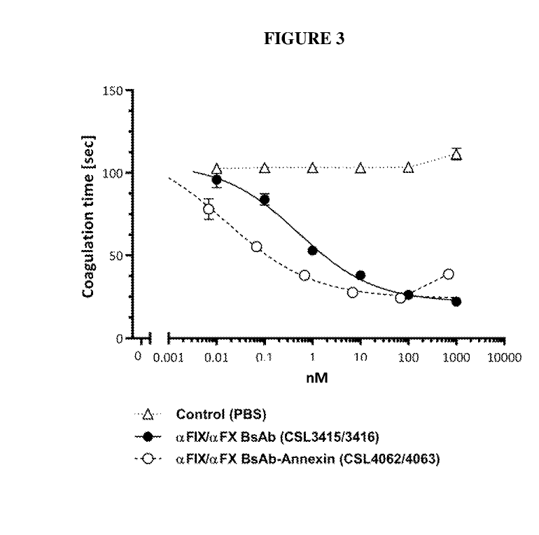

FIG. 3 shows results of five or six independent experiments showing that the membrane targeted anti-Factor IX/X bispecific antibody comprising two Annexin A5 molecules (CSL4062/4063) has increased bypassing activity (EC₅₀ 0.016 nM) compared to the non-membrane targeted anti-Factor IX/X bispecific antibody CSL3415/3416 (EC₅₀ 0.505 nM).

Additionally, membrane targeted anti-Factor IX/X bispecific antibodies comprising only one Annexin A5 molecule have increased FVIII bypassing activity compared to the non-targeted anti-FIX/FX bispecific antibody CSL3415/3416 (EC₅₀ of 0.505 nM). For example, CSL4062/3572, which has an Annexin A5 molecule linked to the anti-FIX heavy chain, had an EC₅₀ of 0.015 nM. CSL3535/4063, which has an Annexin A5 molecule linked to the anti-Factor X heavy chain had an EC₅₀ of 0.015 nM. Four to six independent experiments were performed.

### Example 5: Membrane Targeted Anti-Factor IX Antibodies have Increased Factor VIII Bypassing Activity and Reduced Anti-Coagulant Effect

Expression constructs were generated using standard molecular biology methods. Nucleotide sequences encoding the antibodies, Annexin A5 (NP_001145; SEQ ID NO: 14), E5 mutant of Annexin A5 (SEQ ID NO: 26), truncated Annexin A1 (SEQ ID NO: 30) and GS linker (SEQ ID NO: 20) were synthesized by Geneart (Thermo Fisher Scientific, NY, USA). Sequences were amplified by PCR, digested by restriction and cloned into expression vector by T4 DNA ligase.

Antibodies were generated according to Table 2 below using methods described in Example 1.

TABLE 2
Recombinant antibodies

| Antibody | SEQ ID NO |
|---|---|
| Annexin A5 linked monospecific | Light chain - SEQ ID NO: 1 |
| anti-FIX antibody (CSL4060) | Heavy chain - SEQ ID NO: 8 |
| E5 mutant of Annexin A5 linked | Light chain - SEQ ID NO: 1 |
| monospecific anti-FIX antibody | Heavy chain - SEQ ID NO: 31 |
| Truncated Annexin A1 linked | Light chain - SEQ ID NO: 1 |
| monospecific anti-FIX antibody | Heavy chain - SEQ ID NO: 32 |
| (ATG17090) | |

Factor VIII bypassing activity was measured using the aPTT assay as described above. To fit values to a curve and permit calculation of EC₅₀ values, the highest concentration of the antibody was omitted.

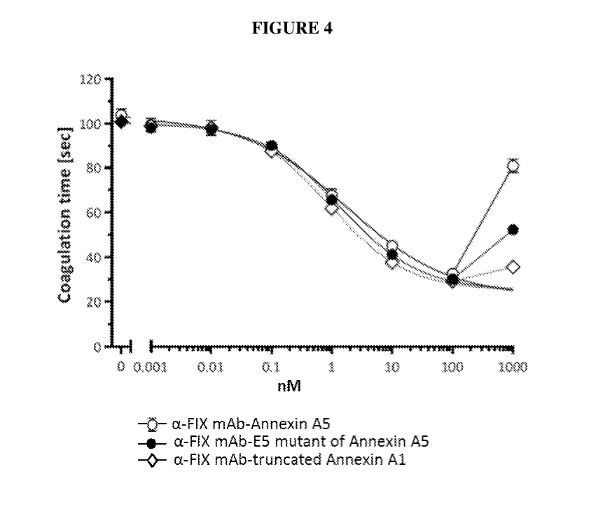

As shown in FIG. 4, the truncated Annexin A1 membrane targeted full anti-Factor IX monospecific antibody has increased bypassing activity (EC₅₀ 0.96 nM) compared to the E5 mutant of Annexin A5 membrane targeted full anti-Factor IX antibody (EC₅₀ 1.39 nM) and the Annexin A5 membrane targeted full anti-Factor IX monospecific antibody (CSL4060; EC₅₀ 1.76 nM). At the highest concentration, the coagulation times for the truncated Annexin A1 anti-FIX antibody, E5 mutant of Annexin A5 anti-FIX antibody and the Annexin A5 anti-FIX antibody were 35.5 seconds (SD 0.3 seconds), 52.3 seconds (SD 3.4 seconds) and 81.0 seconds (3.0 seconds) respectively.

### Example 6: Membrane Targeted Anti-Factor IX Antibodies have FVIII Bypassing Activity in the Presence of Plasma

Male FVIII knockout mice (n=3/group) were treated intravenously with a) 80 IU/kg recombinant Factor IX (BeneFIX®) alone, b) 80 IU/kg recombinant Factor IX (BeneFIX®) and 800 μg/kg of CSL4060, or c) 80 IU/kg recombinant Factor IX (BeneFIX®) and 800 μg/kg BM4-Annexin A5. At approximately 15 minutes post administration, mice were terminally bled using sodium-citrate as anticoagulant (1 part sodium citrate+9 parts whole blood).

Factor VIII bypassing activity was measured in each sample using the aPTT assay as described above and in a one-stage clotting assay using human FVIII depleted plasma (Siemens Healthcare) and Pathromtin SL as activating reagent with the BCS XP (Siemens Healthcare).

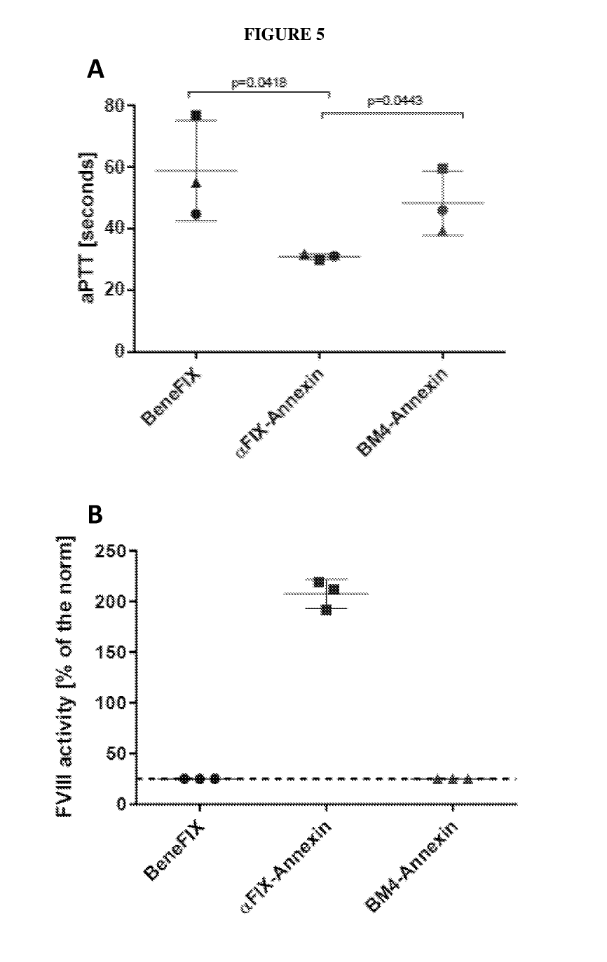

FIG. 5 and Table 3 below shows that samples derived from mice treated with the Annexin A5 membrane-targeted anti-human Factor IX antibody (CSL4060) and human FIX had significantly increased FVIII bypassing activity in the aPTT assay (A) and in the one-stage clotting assay (B) compared to the samples derived from mice treated with recombinant Factor IX alone (p=0.0418 in the aPTT assay) and samples derived from mice treated with Annexin A5 conjugated BM4 antibody and human FIX (p=0.0443 in the aPTT assay).

TABLE 3
FVIII bypassing activity

| | | αFIX- | |
|---|---|---|---|
| | BeneFIX | Annexin A5 | BM4-Annexin A5 |
| aPTT assay | 58.8 (44.7-76.7) | 30.8 (29.8-31.7) | 48.2 (39.2-59.5) |
| (mean; range) | | | |
| One-stage | 5.0 (5.0-5.0) | 41.5 (38.3-43.8) | 5.0 (5.0-5.0) |
| clotting assay | | | |
| (mean; range) | | | |

### Example 7: In Vitro FVIII-Bypassing Activity of Membrane Targeted Anti-Factor IX Antibodies

Factor VIII-bypassing activity of the Annexin A5 membrane-targeted full anti-Factor IX monospecific antibody (CSL4060) was measured in a chromogenic assay in the absence of phospholipids.

Annexin A5 membrane-targeted full anti-Factor IX monospecific antibody (CSL4060), a monospecific anti-FIX antibody (CSL3492), an anti-Factor IX/X bispecific antibody (CSL3415/3416) or BM4 antibody control were pre-mixed with human FIXa and human FX in assay buffer in the absence of phospholipids. Calcium and chromogenic substrate was added to each solution and following cessation of the chromogenic reaction, the Factor VIII bypassing activity of the binding protein was assessed.

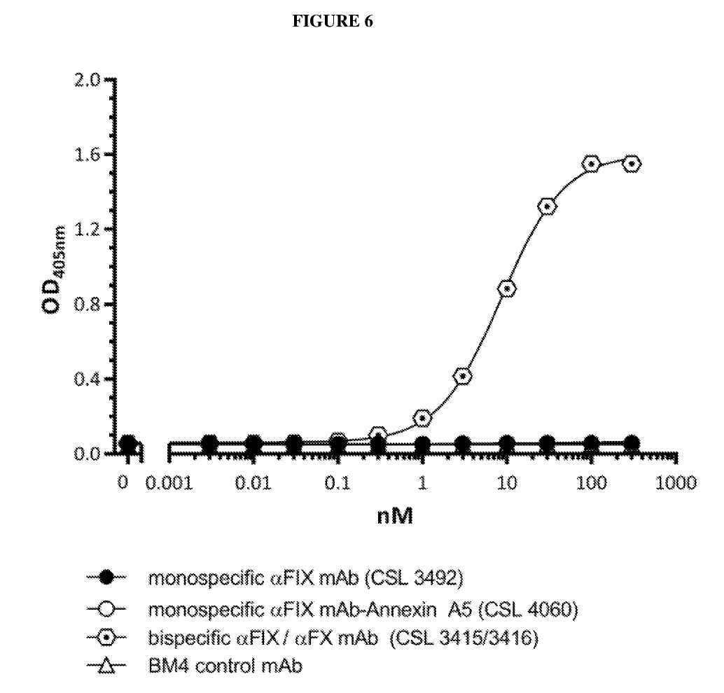

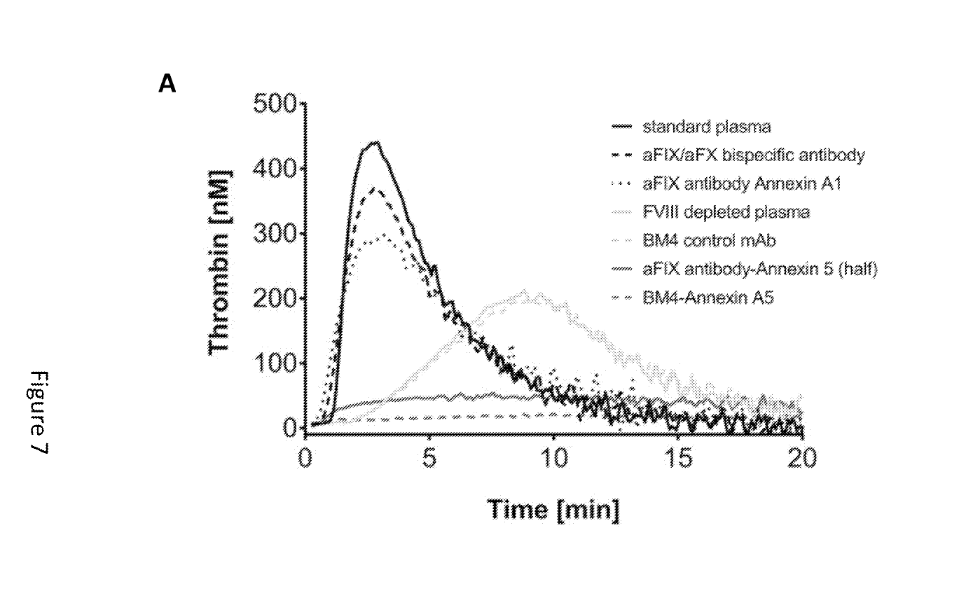

As shown in FIG. 6 and below in Table 4, no Factor VIII bypassing activity was detected with the Annexin A5 membrane-targeted full anti-Factor IX monospecific antibody (CSL4060), monospecific anti-FIX antibody (CSL3492) or BM4 control. In contrast, the anti-Factor IX/X bispecific antibody (CSL3415/3416) displayed Factor VIII bypassing activity in the absence of membranes with an EC₅₀ of 8.6 nM.

TABLE 4
In vitro Factor VIII bypassing activity

| Sample | Mean peak height (range) | Mean Lagtime (range) |
|---|---|---|
| Standard plasma | 450.5 (439.9-455.9) | 1.2 (1.1-1.3) |
| FVIII depleted plasma | 187.7 (137.4-221.7) | 3.4 (3.0-3.7) |
| aFIX/aFX bispecific | 369.3 (360.9-379.1) | 1.0 (1.0-1.2) |
| antibody | | |
| aFIX antibody- | 50.9 (49.1-53.5) | 0.5 (0.3-0.6) |
| Annexin 5 (half) | | |
| aFIX antibody | 312.0 (293.3-328.7) | 0.7 (0.6-0.8) |
| Annexin A1 | | |
| BM4-Annexin A5 | 21.4 (0-48.6) | 1.5 (0-2.4) |
| BM4 | 181.7 (122.5-227.4) | 3.2 (2.9-3.6) |

### Example 8: Relative Activity of Annexin A5- and Truncated Annexin A1-Conjugated Anti-Factor IX Antibodies in a Thrombin Generation Assay

The relative activity of Annexin A5- and truncated Annexin A1-conjugated anti-Factor IX antibodies was measured in an assay designed to measure thrombin generated via the intrinsic coagulation pathway.

Thrombin generation parameters were determined in human FVIII depleted plasma using a calibrated automated thrombogram (CAT). Truncated Annexin A1 membrane-targeted full anti-Factor IX monospecific antibody (ATG17090), Annexin A5 membrane-targeted half anti-Factor IX monospecific antibody (ATG16028), an anti-Factor IX/X bispecific antibody (CSL3415/3416), Annexin A5 conjugated BM4 or BM4 antibody alone were added at concentration of 10 μg/mL to FVIII depleted plasma with a residual of <0.01 U/mL FVIII. Standard human plasma (Siemens Healthcare) served as control. Intrinsic coagulation was triggered by adding of 5 μL RD-reagent.

Briefly, 5 μL of the RD-reagent or thrombin calibrator and 80 μL spiked plasma were pipetted into the wells of a 96-well microplate. The plates were incubated for approximately 10 min at 37° C. on a fluorometer (Fluoroskan Ascent, Thermo Fisher Scientific, Germany). The assay was started by adding 20 μL of fluorogenic substrate into each sample and thrombin calibrator well of the microtiter plate followed by 2 seconds of shaking. Thrombin generated during the assay converted the fluorogenic substrate and changes in fluorescence were recorded in 5 second intervals for a total assay time of one hour. The molar concentration of thrombin generation was calculated based on the respective thrombin calibrator of each sample.

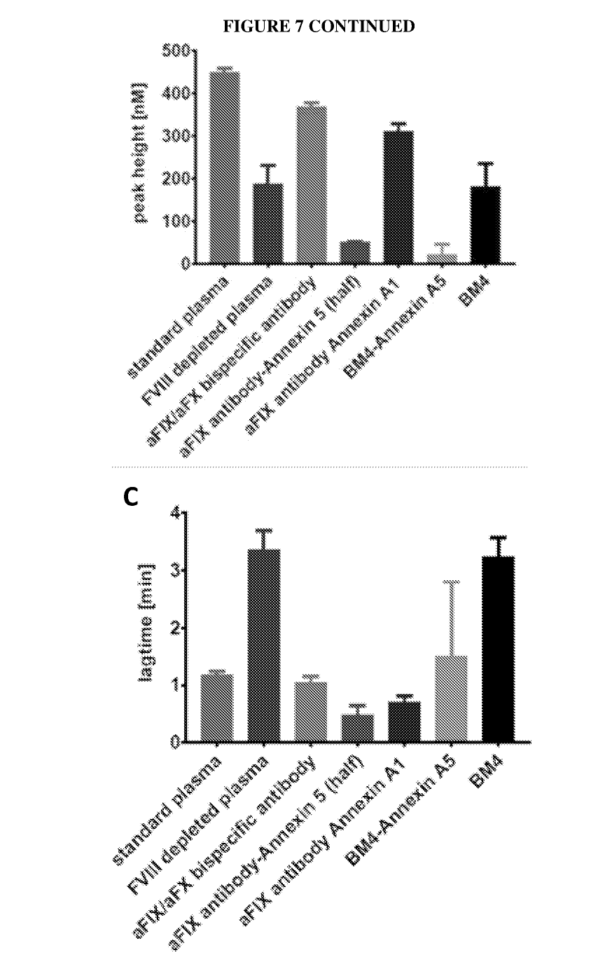

As shown in FIG. 7, thrombin was generated quicker and for longer in FVIII-depleted plasma in the presence of 10 μg/mL of an anti-Factor IX/X bispecific antibody and an Annexin A1 membrane-targeted full anti-Factor IX monospecific antibody compared to an Annexin A5 membrane-targeted anti-Factor IX half antibody.

### Example 9: Increased FVIII Bypassing Activity of Additional Membrane Targeted Anti-Factor IX Antibodies

Anti-Factor IX monospecific antibodies A10, B2 and C12 and Annexin A5 conjugated versions thereof were generated (Table 5) and their relative Factor VIII-bypassing activity was measured using the chromogenic FVIII bypassing assay as previously described.

TABLE 5
Affinity matured antibodies

| Antibody | SEQ ID NO |
|---|---|
| Annexin A5 linked monospecific | Light chain - SEQ ID NO: 1 |
| anti-FIX antibody A10 | Heavy chain - SEQ ID NO: 36 |
| Annexin A5 linked monospecific | Light chain - SEQ ID NO: 1 |
| anti-FIX antibody B2 | Heavy chain - SEQ ID NO: 39 |
| Annexin A5 linked monospecific | Light chain - SEQ ID NO: 1 |
| anti-FIX antibody C12 | Heavy chain - SEQ ID NO: 42 |

As shown in Table 6 below, the three Annexin A5 membrane-targeted affinity matured anti-Factor IX monospecific antibodies (A10, B2 and C12) had improved FVIII bypass activity compared to their unconjugated counterparts at 10 nM concentration in at least three independent experiments.

TABLE 6
FVIII bypass activity of affinity matured membrane-targeted antibodies

| | Mean OD_{405 nm} |
|---|---|
| | (range) at 10 nM |
| Affinity matured monospecific anti-FIX antibody | 0.095 (0.090-0.107) |
| A10 | |
| Annexin A5 linked affinity matured monospecific | 0.126 (0.118-0.133) |
| anti-FIX antibody A10 | 1.3-fold increase |
| Affinity matured monospecific anti-FIX antibody | 0.102 (0.092-0.119) |
| B2 | |
| Annexin A5 linked affinity matured monospecific | 0.246 (0.224-0.272) |
| anti-FIX antibody B2 | 2.4-fold increase |
| Affinity matured monospecific anti-FIX antibody | 0.091 (0.084-0.104) |
| C12 | |
| Annexin A5 linked affinity matured monospecific | 0.119 (0.110-0.129) |
| anti-FIX antibody C12 | 1.3-fold increase |

1) A membrane targeted binding protein that binds to at least one blood coagulation factor, wherein the binding protein has pro-coagulant activity.

2) The protein of claim 1, wherein the protein comprises a first binding region that specifically binds to the blood coagulation factor and a second binding region that specifically binds to a component of a plasma membrane of a mammalian cell, wherein the first binding region has pro-coagulant activity.

3. (canceled)

4. The protein of claim 2 or 3, wherein the first and/or second binding region comprises:
(i) a single chain Fv fragment (scFv);
(ii) a dimeric scFv (di-scFv);
(iii) a diabody;
(iv) a triabody;
(v) a tetrabody;
(vi) a Fab;
(vii) a F(ab′)2;
(viii) a Fv;
(ix) one of (i) to (viii) linked to a constant region of an antibody, Fc or a heavy chain constant domain (CH) 2 and/or CH3; or
(x) an antibody or antigen binding fragment thereof, an antibody mimetic, a domain antibody, a chimeric antibody or a fusion protein.

5. (canceled)

6. The protein of claim 2, wherein the first binding region is monospecific, bispecific, or multispecific.

7. The protein of claim 1, wherein the blood coagulation factor is selected from the group consisting of Factor I, Factor II (prothrombin)/thrombin, Factor III, Factor V, Factor VII, Factor VIII, Factor IX, Factor X, Factor XI, Factor XII Factor XIII and an activated form of any of the foregoing.

8. The protein of claim 2, wherein the first binding region is an anti-Factor IX antibody or Factor IX binding fragment thereof, wherein the anti-Factor IX antibody or Factor IX binding fragment thereof binds to non-activated Factor IX (FIX) and/or activated Factor IX (FIXa).

9. (canceled)

10. The protein of claim 2, wherein the first binding region specifically binds to factor IX/IXa and factor X/Xa.

11. The protein of claim 4, wherein the first binding region is a half antibody.

12. The protein of claim 4, wherein the first binding region antigen comprises an IgG₄ constant region or a stabilised IgG4 constant region, wherein the IgG₄ constant region comprises a sequence set forth in any one of SEQ ID NO: 15 to SEQ ID NO: 19.

13. (canceled)

14. The protein of claim 2, wherein:
(a) the first binding region comprises a VH comprising a sequence set forth in any one of SEQ ID NO: 2 to 7, SEQ ID NO: 34, SEQ ID NO: 37 or SEQ ID NO: 40 and a VL comprising a sequence set forth in SEQ ID NO: 1; and/or
(b) the protein comprises:
(i) a V_{L} sequence set forth in SEQ ID NO: 1, a V_{H} sequence set forth in SEQ ID NO: 2 and a V_{H} sequence set forth in SEQ ID NO: 3; or
(ii) a V_{L} sequence set forth in SEQ ID NO: 1 and a V_{H} sequence set forth in SEQ ID NO: 4; or
(iii) a V_{L} sequence set forth in SEQ ID NO: 1 and a V_{H} sequence set forth in SEQ ID NO: 5; or
(iv) a V_{L} sequence set forth in SEQ ID NO: 1 and a V_{H} sequence set forth in SEQ ID NO: 6; or
(v) a V_{L} sequence set forth in SEQ ID NO: 1 and a V_{H} sequence set forth in SEQ ID NO: 7; or
(vi) a V_{L} sequence set forth in SEQ ID NO: 1 and a V_{H} sequence set forth in SEQ ID NO: 8; or
(vii) a V_{L} sequence set forth in SEQ ID NO: 1 and a V_{H} sequence set forth in SEQ ID NO: 9; or
(viii) a V_{L} sequence set forth in SEQ ID NO: 1 and a V_{H} sequence set forth in SEQ ID NO: 10; or
(ix) a V_{L} sequence set forth in SEQ ID NO: 1, a V_{H} sequence set forth in SEQ ID NO: 31; or
(x) a V_{L} sequence set forth in SEQ ID NO: 1, a V_{H} sequence set forth in SEQ ID NO: 32; or
(xi) a V_{L} sequence set forth in SEQ ID NO: 1, a V_{H} sequence set forth in SEQ ID NO: 33;
(xii) a V_{L} sequence set forth in SEQ ID NO: 1, a V_{H} sequence set forth in SEQ ID NO: 34; or
(xiii) a V_{L} sequence set forth in SEQ ID NO: 1, a V_{H} sequence set forth in SEQ ID NO: 36; or
(xiv) a V_{L} sequence set forth in SEQ ID NO: 1, a V_{H} sequence set forth in SEQ ID NO: 37; or
(xv) a V_{L} sequence set forth in SEQ ID NO: 1, a V_{H} sequence set forth in SEQ ID NO: 39; or
(xvi) a V_{L} sequence set forth in SEQ ID NO: 1, a V_{H} sequence set forth in SEQ ID NO: 40; or
(xvii) a V_{L} sequence set forth in SEQ ID NO: 1, a V_{H} sequence set forth in SEQ ID NO: 42; or
(xviii) a V_{L} sequence set forth in SEQ ID NO: 1, a V_{H} sequence set forth in SEQ ID NO: 9 and a V_{H} sequence set forth in SEQ ID NO: 10; or
(xix) a V_{L} sequence set forth in SEQ ID NO: 1, a V_{H} sequence set forth in SEQ ID NO: 6 and a V_{H} sequence set forth in SEQ ID NO: 10; or
(xx) a V_{L} sequence set forth in SEQ ID NO: 1, a V_{H} sequence set forth in SEQ ID NO: 9 and a V_{H} sequence set forth in SEQ ID NO: 7; or
(xxi) a V_{L} sequence set forth in SEQ ID NO: 1, a V_{H} sequence set forth in SEQ ID NO: 54; or
(xxii) a V_{L} sequence set forth in SEQ ID NO: 1, a V_{H} sequence set forth in SEQ ID NO: 55; and/or
(c) the first binding region is a half antibody comprising:
(i) a V_{H} comprising:
(a) a CDR1 comprising a sequence set forth in amino acids 31 to 35 of SEQ ID NO: 13;
(b) a CDR2 comprising a sequence set forth in amino acids 50 to 66 of SEQ ID NO: 13; and
(c) a CDR3 comprising a sequence set forth in amino acids 99 to 112 of SEQ ID NO: 13; and
(ii) a V_{L} comprising:
(a) a CDR1 comprising a sequence set forth in amino acids 24 to 34 of SEQ ID NO: 11;
(b) a CDR2 comprising a sequence set forth in amino acids 50 to 56 of SEQ ID NO: 11; and
(c) a CDR3 comprising a sequence set forth in amino acids 89 to 97 of SEQ ID NO: 11.

15. (canceled)

16. (canceled)

17. The protein of claim 2, wherein the component of the plasma membrane is selected from the group consisting of an aminophospholipid; a membrane-associated polypeptide, and mixtures thereof, wherein:
(a) the aminophospholipid is selected from the group consisting of a phosphatidylserine, a phosphatidylethanolamine and mixtures thereof; and/or
(b) the membrane-associated polypeptide is selected from the group consisting of GPIIb/IIIa, β2GP1, TLT-1, a coagulation factor, a selectin and mixtures thereof.

18. (canceled)

19. (canceled)

20. The protein of claim 2, wherein the second binding region is selected from the group consisting of an antibody or antigen binding fragment thereof, an annexin or variant, a gamma-carboxyglutamic acid-rich (GLA) domain or variant, a lactadherin domain, a PSP1 peptide or variant, a protein kinase C (PKC) domain and a pleckstrin homology domain, wherein the annexin is Annexin A5 comprising a sequence set forth in SEQ ID NO: 14 or a truncated Annexin A1 comprising a sequence set forth in SEQ ID NO: 30 or a phosphatidylserine binding fragment or variant of Annexin A5 or a phosphatidylserine binding fragment or variant of Annexin A1.

21. (canceled)

22. (canceled)

23. The protein of claim 2, wherein the first binding region is linked to the second binding region via a linker, wherein the linker is a peptide linker comprising between 2 and 31 amino acids in length.

24. (canceled)

25. The protein of claim 1, wherein:
(a) the protein comprises:
(i) a first binding region comprising an anti-Factor IX antibody or Factor IX binding fragment thereof; and
(ii) a second binding region comprising:
(a) Annexin A5 comprising a sequence set forth in SEQ ID NO: 14; or
(b) truncated Annexin A1 comprising a sequence set forth in SEQ ID NO: 30;
(c) a phosphatidylserine binding fragment or variant of Annexin A5; or
(d) a phosphatidylserine binding fragment or variant of Annexin A1_{L} or
(b) the protein comprises:
(i) a first binding region that is an anti-Factor IX/IXa half antibody comprising a V_{H} comprising a sequence set forth in SEQ ID NO: 13 and a V_{L} comprising a sequence set forth in SEQ ID NO: 11; and
(ii) a second binding region comprising Annexin A5 comprising a sequence set forth in SEQ ID NO: 14.

26. The protein of claim 25, wherein a linker joins the N-terminus of the second binding region to the C-terminus of a heavy chain or domain thereof or a light chain or domain thereof of the anti-Factor IX antibody or antigen binding fragment thereof.

27. The protein of claim 26, wherein the linker extends between the N-terminus of the second binding region and the C-terminus of a heavy chain or domain thereof of the anti-Factor IX antibody or antigen binding fragment thereof.

28. (canceled)

29. A composition comprising the protein of claim 1 and a pharmaceutically acceptable carrier.

30. A method of treating or preventing a disease or condition in a subject, the method comprising administering the protein of claim 1 to a subject in need thereof, wherein the disease or condition is a bleeding disorder selected from the group consisting of haemophilia A, haemophilia B, von Willebrand disease, Factor I deficiency, Factor II deficiency, Factor V deficiency, combined Factor V/Factor VIII deficiency, Factor VII deficiency, Factor X deficiency, Factor XI deficiency and Factor XIII deficiency.

31. (canceled)

32. (canceled)

33. The method of claim 30, wherein the subject:
(a) is at risk of developing a bleeding disorder or symptoms thereof and/or
(b) suffers from haemophilia A and has developed inhibitors of factor VIII.

34-36. (canceled)

37. A method of treating or preventing a disease or condition in a subject, the method comprising administering the composition of claim 29 to a subject in need thereof, wherein the disease or condition is a bleeding disorder selected from the group consisting of haemophilia A, haemophilia B, von Willebrand disease, Factor I deficiency, Factor II deficiency, Factor V deficiency, combined Factor V/Factor VIII deficiency, Factor VII deficiency, Factor X deficiency, Factor XI deficiency and Factor XIII deficiency.
